(19) United States
(12) Patent Application Publication
(10) Pub. No.: US 2026/0062694 A1
(43) Pub. Date: Mar. 5, 2026

(54) SYSTEMS AND METHODS TO IMPROVE NUCLEIC ACID SYNTHESIS AND PRODUCTION

(71) Applicant: GE Precision Healthcare LLC, Waukesha, WI (US)

(72) Inventors: John Richard Nelson, Clifton Park, NY (US); Erik Leeming Kvam, Niskayuna, NY (US); Weston Blaine Griffin, Niskayuna, NY (US); Alex David Corwin, Niskayuna, NY (US); Zhen Liu, Waterford, NY (US); Wei Gao, Norristown, PA (US); Robert Martin Martinez, Schenectady, NY (US)

(21) Appl. No.: 18/819,115

(22) Filed: Aug. 29, 2024

(51) Int. Cl. C12N 15/10

(52) U.S. Cl. CPC C12N 15/1003

(57) ABSTRACT

A system for purifying a deoxyribonucleic acid (DNA) product from a rolling circle amplification (RCA) reaction includes a DNA purification module configured to perform actions. The actions include obtain a reaction volume having the DNA product from the RCA reaction. The actions also include diluting the reaction volume with a buffer to form a diluted reaction volume. The actions further include flowing of the diluted reaction volume through a first purification process configured to remove protein to generate a protein-depleted DNA product. The actions even further include flowing the protein-depleted DNA product through a second purification process configured to positively select for the DNA product using anion exchange. The actions further include eluting a purified DNA product from the second purification process.

### STATEMENT REGARDING FEDERALLY SPONSORED RESEARCH & DEVELOPMENT

This invention was made with US Government support under contract number N6600121-C-4014 awarded by Defense Advanced Research Projects Agency. The Government has certain rights in the invention.

### BACKGROUND

The subject matter disclosed herein relates to systems and methods for improving nucleic acid synthesis.

Production of nucleic acid-based medical therapeutics for military and humanitarian purposes is impaired by the requirements of current workflows that parse together multiple manual, labor intensive, steps which include the use of reaction vessels (e.g., bioreactors) and resulting intense purification, and the production of significant volumes of chemical waste. The workflow takes months, and production takes place at centralized good manufacturing practices (GMP) facilities far from the chemical, biological, radiological, and nuclear (CBRN) point of need. Manufacturing of high-quality nucleic acid requires removal of protein components that could otherwise serve as antigens if preserved in medical countermeasure injectables and co-administered with the nucleic acid. Therefore, systems and methods for efficiently removing protein components from nucleic acid are desired, especially for functionally-closed manufacturing processes. Monitoring of nucleic acid quality and quantity throughout the manufacturing processes, without compromising the functionally-closed property, are also desired to meet high end product quality standards required for the medical countermeasures for military and humanitarian purposes.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a system is provided. The system includes a nucleic acid amplification module configured to receive deoxyribonucleic acid (DNA) template and to generate a nucleic acid product from the DNA template utilizing an amplification reaction while performing real-time inline monitoring of the amplification reaction via a plurality of sensors. The system also includes a purification module configured to purify the nucleic acid product. The nucleic acid amplification module and the purification module are each automated and form a functionally closed system.

In another embodiment, a method is provided. The method includes receiving circular deoxyribonucleic acid (DNA) template at a nucleic acid amplification module. The method also includes generating, via the nucleic acid amplification module, an amplified DNA product from the circular DNA template utilizing an amplification reaction, wherein the nucleic acid amplification module includes a pump configured to cause flow of a portion of the amplification reaction throughout a quality control panel. The method further includes performing real time inline monitoring of the amplification reaction at the quality control panel via a plurality of sensors in communication with a controller having a memory and a processor, wherein the controller is configured to receive feedback from the plurality of sensors. The method even further includes ceasing, in response to control signals from the controller, the amplification reaction and providing the amplified product to a purification module upon reaching a defined quality range and generating a purified nucleic acid product.

In a further embodiment, a non-transitory computer-readable medium is provided. The computer-readable medium includes processor-executable code that, when executed by a processing system, causes the processing system to perform actions. The actions include providing control signals to a nucleic acid amplification module to generate an amplified product from circular deoxyribonucleic acid (DNA) template utilizing a rolling circle amplification reaction under isothermal conditions. The actions also include receiving feedback from a plurality of sensors performing real time inline measurements of kinetics and conditions of the rolling circle amplification reaction. The actions further include providing control signals based on the feedback to regulate purification of the rolling circle amplification reaction.

In one embodiment, a system is provided. The system includes a hydration module configured to rehydrate lyophilized reagents for a rolling circle amplification reaction. The system also includes a deoxyribonucleic acid (DNA) amplification module configured to generate a DNA product from a DNA template and rehydrated reagents utilizing a rolling circle amplification reaction.

In another embodiment, a method is provided. The method includes storing lyophilized reagents for a rolling circle amplification reaction on a deoxyribonucleic acid (DNA) amplification module. The method also includes automatically utilizing a hydration system to rehydrate the lyophilized reagents prior to use in the rolling circle amplification reaction. The method further includes automatically generating a DNA product from circular DNA template via the rolling circle amplification reaction in a reaction vessel of the DNA amplification module utilizing the lyophilized reagents that have been rehydrated.

In a further embodiment, a non-transitory computer-readable medium is provided. The computer-readable medium includes processor-executable code that, when executed by a processing system, causes the processing system to perform actions. The actions include automatically utilize a hydration system to rehydrate lyophilized reagents for a rolling circle amplification reaction prior to use in the rolling circle amplification reaction, wherein the lyophilized reagents are stored on a deoxyribonucleic acid (DNA) amplification module. The actions also include automatically utilize a hydration system to rehydrate lyophilized reagents for a rolling circle amplification reaction prior to use in the rolling circle amplification reaction, wherein the lyophilized reagents are stored on a deoxyribonucleic acid (DNA) amplification module.

In one embodiment, a method for producing purified deoxyribonucleic acid (DNA) is provided. The method includes providing a circular DNA template. The method also includes amplifying in vitro the circular DNA template to generate a DNA product that is rolling circle amplified. The method further includes purifying the DNA product utilizing at least one chromatographic step to obtain a protein-depleted purified DNA product.

In another embodiment, a method for producing purified deoxyribonucleic acid (DNA) from a rolling circle amplification reaction is provided. The method includes providing a circular DNA template. The method also includes amplifying in vitro the circular DNA template to generate a DNA product. The method further includes purifying the DNA product utilizing at least one chromatographic step to obtain a protein-depleted purified DNA product. The method even further includes assessing a quality of either the DNA product prior to purification or the purified DNA product by respectively determining a viscosity of either the DNA product prior to purification or the protein-depleted purified DNA product after purification.

In a further embodiment, a method for producing purified deoxyribonucleic acid (DNA) from a rolling circle amplification reaction is provided. The method includes providing a circular DNA template. The method also includes amplifying in vitro the circular DNA template to generate a DNA product. The method further includes purifying the DNA product utilizing at least one chromatographic step to obtain a protein-depleted purified DNA product. The method even further includes assessing a quality of the purified protein-depleted DNA product by determining if protein is present in a sample of the purified protein-depleted DNA product.

In one embodiment, a method for monitoring generation of a nucleic acid is provided. The method includes conducting in vitro an amplification reaction or a synthesis reaction to generate the nucleic acid. The method also includes directly monitoring amplification reaction kinetics or synthesis reaction kinetics in real time utilizing a sensor configured to measure an absorbance at a wavelength of between 230 to 285 nanometers utilizing a minimal optical path length.

In another embodiment, a system for monitoring generation of a nucleic acid is provided. The system comprises a reaction vessel and connected tubing configured for conducting in vitro an amplification reaction or a synthesis reaction to generate the nucleic acid. The system also includes a first sensor configured to directly measure an amplification reaction or a synthesis reaction at an absorbance at a wavelength of between 230 to 285 nanometers utilizing a minimal optical path length or a second sensor configured to directly measure the amplification reaction or the synthesis reaction at an absorbance at a wavelength of between 295 to 310 nanometers. The system further includes a controller having a memory and a processor. The controller is configured to receive feedback from the first sensor or the second sensor and to directly monitor amplification reaction kinetics or synthesis reaction kinetics in real time based on the feedback.

In a further embodiment, a non-transitory computer-readable medium, the computer-readable medium including processor-executable code that, when executed by a processing system, causes the processing system to perform actions. The actions include conducting in vitro an amplification reaction or a synthesis reaction to generate a nucleic acid. The actions also include directly monitoring amplification reaction kinetics or synthesis reaction kinetics in real time utilizing a sensor configured to measure an absorbance at a wavelength of 260 nanometers utilizing a minimal optical path length or to measure an absorbance at a wavelength between 295 to 310 nanometers.

In another embodiment, a method for monitoring generation of a nucleic acid is provided. The method includes conducting in vitro an amplification reaction or a synthesis reaction to generate the nucleic acid. The method also includes directly monitoring amplification reaction kinetics or synthesis reaction kinetics in real time utilizing a sensor configured to measure an absorbance at a wavelength of between 294 to 310 nanometers.

In one embodiment, a system for purifying a deoxyribonucleic acid (DNA) product from a rolling circle amplification (RCA) reaction is provided. The system includes a DNA purification module configured to perform actions. The actions include obtain a reaction volume having the DNA product from the RCA reaction. The actions also include diluting the reaction volume with a buffer to form a diluted reaction volume. The actions further include flowing of the diluted reaction volume through a first purification process configured to remove protein to generate a protein-depleted DNA product. The actions even further include flowing the protein-depleted DNA product through a second purification process configured to positively select for the DNA product using anion exchange. The actions further include eluting a purified DNA product from the second purification process.

In another embodiment, a method for purifying a deoxyribonucleic acid (DNA) product from a rolling circle amplification (RCA) reaction is provided. The method includes obtaining a reaction volume having the DNA product from the RCA reaction. The method also includes diluting the reaction volume with a buffer to form a diluted reaction volume. The method further includes flowing the diluted reaction buffer through a first chromatographic purification process configured to remove protein and to generate a protein-depleted DNA product. The protein-depleted DNA is further flowed through a second purification process configured to positively select for the DNA product using anion exchange. The method further includes eluting a purified DNA product from the second purification process.

In a further embodiment, non-transitory computer-readable medium. The computer-readable medium includes processor-executable code that, when executed by a processing system, causes the processing system to perform actions. The actions include obtaining a reaction volume having a deoxyribonucleic acid (DNA) product from a rolling circle amplification reaction. The actions also include diluting the reaction volume with a buffer to form a diluted reaction volume. The actions further include flowing the diluted reaction buffer through a first chromatographic purification process configured to remove protein and generate a protein-depleted DNA product. The actions further include flowing the protein-depleted DNA product through a second purification process configured to positively select for the DNA product using anion exchange. The actions further include eluting a purified DNA product from the second purification process.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIGS. 1A and 1B are schematic diagrams of a system for generating nucleic acids and associated workflows, in accordance with certain aspects of the present disclosure;

FIG. 2 is a schematic diagram of a DNA amplification module utilized for a single construct large mass system, in accordance with certain aspects of the present disclosure;

FIG. 3 is a schematic diagram of a DNA amplification module utilized for a multi-construct, low mass system, in accordance with certain aspects of the present disclosure;

FIG. 4 is a schematic diagram of a purification module, in accordance with aspects of the present disclosure;

FIG. 5 is a schematic diagram of a fill-finish module, in accordance with certain aspects of the present disclosure;

FIG. 6 is a schematic diagram of the system in FIG. 1A and FIG. 1B, in accordance with certain aspects of the present disclosure;

FIG. 7 is a schematic diagram of a user interfacing with the system in FIG. 1A and FIG. 1B, in accordance with certain aspects of the present disclosure;

FIG. 8 is a flow chart of a method for generating and purifying nucleic acid, in accordance with aspects of the present disclosure;

FIG. 9 is a schematic diagram of a first scenario for the storage, rehydration, and utilization of the lyophilized reagents, in accordance with certain aspects of the present disclosure;

FIG. 10 is a schematic diagram of a second scenario for the storage, rehydration, and utilization of the lyophilized reagents, in accordance with certain aspects of the present disclosure;

FIG. 11 is a schematic diagram of a third scenario for the storage, rehydration, and utilization of the lyophilized reagents, in accordance with certain aspects of the present disclosure;

FIG. 12 is a schematic diagram of a fourth scenario for the storage, rehydration, and utilization of the lyophilized reagents, in accordance with certain aspects of the present disclosure;

FIG. 13 is a schematic diagram of a fifth scenario for the storage, rehydration, and utilization of the lyophilized reagents, in accordance with certain aspects of the present disclosure;

FIG. 14 is a schematic diagram of a fluid architecture of an automated DNA amplification module, in accordance with certain aspects of the present disclosure;

FIG. 15 is a schematic diagram of a fluid architecture of an automated DNA amplification module utilizing two-stage bioreactor, in accordance with certain aspects of the present disclosure;

FIG. 16 is a schematic diagram of a fluid architecture of an automated DNA amplification module for a two-stage amplification reaction, in accordance with certain aspects of the present disclosure;

FIGS. 17 and 18 are schematic diagrams of a workflow for automated DNA amplification (e.g., with a two-stage bioreactor), in accordance with certain aspects of the present disclosure;

FIG. 19 is a flow chart of a method for monitoring a DNA amplification reaction, in accordance with certain aspects of the present disclosure;

FIG. 20 depicts graphs of the monitoring of parameters during in different rolling circle amplification reaction conditions, in accordance with certain aspects of the present disclosure;

FIG. 21 depicts a graph of monitoring of pressure during a rolling circle amplification reaction, in accordance with aspects of the present disclosure;

FIG. 22 depicts a graph of monitoring of viscosity during a rolling circle amplification reaction, in accordance with aspects of the present disclosure;

FIG. 23 depicts a graph of monitoring of conductivity during a rolling circle amplification reaction, in accordance with aspects of the present disclosure;

FIG. 24 depicts a graph of monitoring of refractive index during a rolling circle amplification reaction, in accordance with aspects of the present disclosure;

FIG. 25 is a flow chart of a method for monitoring generation of a nucleic acid, in accordance with certain aspects of the present disclosure;

FIG. 26 is a schematic diagram of a workflow for purification of a DNA product, in accordance with aspects of the present disclosure;

FIG. 27 is a schematic diagram of a fluid architecture of a first portion (e.g., for protein removal) of an automated purification module, representing an exemplary single-use kit design for mediating protein removal from RCA DNA within a functionally-closed unit operation, in accordance with aspects of the present disclosure;

FIG. 28 is a schematic diagram of a fluid architecture of a second portion (e.g., for ion exchange purification of DNA prior to product fill-finish) of an automated purification module, representing an exemplary single-use kit design for binding, washing, and eluting concentrated RCA DNA within a functionally-closed unit operation, in accordance with aspects of the present disclosure;

FIG. 29 depicts a graph of monitoring absorbance at 260 nanometers during anionic purification of DNA, in accordance with aspects of the present disclosure;

FIG. 30 is a flow chart of a method for purification of a DNA product, in accordance with certain aspects of the present disclosure;

FIG. 31 depicts the efficiency of phi29 removal from RCA DNA product as a function of resin type and diluent factors, in accordance with aspects of the present disclosure;

FIG. 32 depicts how SDS detergent is both necessary and sufficient for effective phi29 removal from RCA DNA in the presence of EDTA and heparin-immobilized resin, in accordance with aspects of the present disclosure;

FIG. 33 depicts how heparin-immobilized resin is able to remove additional proteins (beyond phi29) from RCA DNA reactions in the presence of EDTA and SDS, in accordance with aspects of the present disclosure;

FIG. 34 depicts that ion exchange materials are capable of selectively purifying RCA DNA in a substantially de-branched state, in accordance with aspects of the present disclosure; and

FIG. 35 depicts offline final yield quantification of DNA products from multiple constructs in the amplification module in FIG. 2, in accordance with aspects of the present disclosure;

FIG. 36 depicts a gel of DNA products generated utilizing the amplification module in FIG. 2 with multiple constructs in accordance with aspects of the present disclosure; and

FIGS. 37A-37C depict graphs of real time monitoring of RCA reactions under different conditions, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

The term processor, processing system, or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the terms “application”, “application module” (or “module”), “engine”, or “program”, or “plugin” refers to one or more sets of computer software instructions (e.g., computer programs and/or scripts) executable by one or more processors of a computing system to provide particular functionality. Computer software instructions can be written in any suitable programming languages, such as C, C++, C#, Pascal, Fortran, Perl, MATLAB, SAS, SPSS, JavaScript, AJAX, Python, and JAVA. Such computer software instructions can comprise an independent application with data input and data display aspects (e.g., modules). Alternatively, the disclosed computer software instructions can be classes that are instantiated as distributed objects. The disclosed computer software instructions can also be component software, for example JAVABEANS or ENTERPRISE JAVABEANS. Additionally, the disclosed applications or engines can be implemented in computer software, computer hardware, or a combination thereof.

As used herein, the terms “automatic” and “automatically” refer to actions that are performed by a computing device or computing system (e.g., of one or more computing devices) without human intervention. For example, automatically performed functions may be performed by computing devices or systems based solely on data stored on and/or received by the computing devices or systems despite the fact that no human users have prompted the computing devices or systems to perform such functions. As but one non-limiting example, the computing devices or systems may make decisions and/or initiate other functions based solely on the decisions made by the computing devices or systems, regardless of any other inputs relating to the decisions.

The present disclosure provides for systems and methods for systems and methods for improving nucleic acid synthesis. In particular, a system and method are provided for manufacturing nucleic acids with real time monitoring of key steps to ensure the output product meets desired quality metrics. The overall system includes multiple functional technology components (e.g., modules) that together function to generate the output product. In certain embodiments, the overall system is configured to be field deployable to a location of need and does not require highly skilled labor or a specialized operating environment. The overall system utilizes an integrated workflow including enzymatic-based biosynthesis of deoxyribonucleic acid (DNA) “seed” template, cell-free amplification of template DNA to rapidly produce DNA, and optional conversion to a ribonucleic acid (RNA) product. In certain embodiments, the final product may be a vaccine. In certain embodiments, the entire workflow includes quality control steps to produce GMP compliant doses and will be integrated within a user-friendly, portable structure that can be activated at a time of need (e.g., at the location of need) to produce hundreds of ready-to-use doses for military or civilian first responder use (e.g., in less than 3 days). In this scenario, the system utilizes utilize a high volume (e.g., liter-scale) to generate bulk quantities of a single molecule (e.g., vaccine) within a functionally-closed unit operation. In certain embodiments, the system may utilize a low volume, high throughput process to generate smaller quantities of DNA products (e.g., different DNA products) for a variety of applications (e.g., screening, etc.). In this low volume, high throughput process (e.g., milliliter—to microliter-scale) parallel reactions occur in respective individual wells of a multi-well plate.

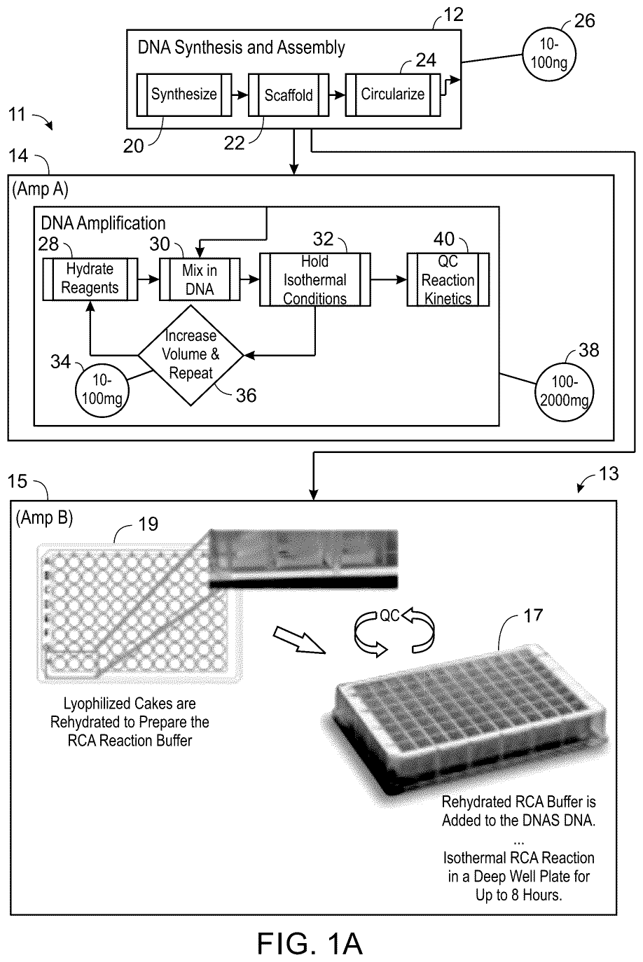

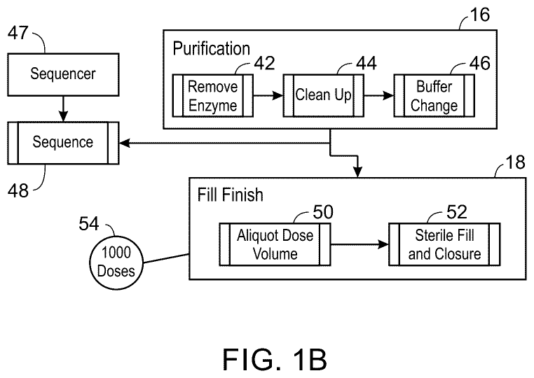

FIGS. 1A and 1B are schematic diagrams of a system 10 for generating nucleic acids and associated workflows (e.g., workflow 11 and workflow 13). It should be noted that even though the system 10 is described with regard to utilizing it to generate DNA, the system 10 may also be utilized to generate RNA (e.g., via in vitro transcription from the generated DNA product). The system 10 includes the following functional modules: optionally DNA synthesis and assembly module 12, either nucleic acid (DNA amplification) module 14 or 15, purification module 16, and fill-finish module 18. The DNA synthesis and assembly module 12 is optionally configured to enzymatically synthesize short DNA templates (e.g., 30 to 120 nucleotides) and to assemble these into gene expression constructs. The DNA amplification modules 14 and 15 are configured to generate (e.g., amplify) a DNA product (e.g., endotoxin free DNA product) from a DNA template. The purification module 16 is configured to purify the DNA product (or nucleic acid product) with real-time inline monitoring of the purification process via a plurality of sensors. The fill-finish module 18 is configured to aliquot the DNA product upon purification into a plurality of doses ready for use.

The DNA amplification module 14 or 15, the purification module 16, and the fill-finish module 18 are each automated and form a functionally closed system (e.g., to minimize contaminants). In a functionally closed system, the product is not exposed to the operations environment. Materials can be introduced into the system; however, it must be done in such a way as to avoid exposing the product. System closure is often defined by the boundary in which the product can be separated from the environment and may be different than the physical boundaries of process equipment. The system may be intrinsically closed in that they are capable of generating or creating a sterile boundary, for instance sterile single-use connectors which do not expose the internal flow path to the environment during assembly. In a connected process, two or more-unit operations are physically connected. The unit operations may be batch, continuous, or a mix. Planned product hold steps, hold-up volumes in between, and manual interventions are minimized or eliminated. Discrete product quality states may exist in between unit operations.

In the system 10, single use consumable kits may be utilized with each module. Also, the system 10 may utilize connectors for reagents and module to module connections. The system 10 may also off system connections for extended modularity or easy sampling for at-line quality control (e.g., sequencing). The modularity of the system 10 may also enable asynchronous batch processing. For example, a kit can be swapped on a module while another module is running. In addition, the system 10 may allow just-in-time connections of reagents so that expensive reagents are not exposed/consumed if a prior operation (at an upstream module) fails.

As depicted in FIG. 1, in certain embodiments, the DNA synthesis and assembly module 12 is optionally configured to synthesize DNA templates as depicted by reference numeral 20. For example, the DNA templates may be generated chemically or enzymatically synthesized. Those familiar with the art would recognize the general steps for creating DNA chemically or enzymatically. In certain embodiments, the DNA synthesis and assembly module 12 may not synthesize DNA but instead provide a circular plasmid template. The DNA synthesis and assembly module 12 is also configured to perform scaffolding (e.g., assembly) as depicted by reference numeral 22. In particular, the oligomers are ligated together (e.g., assembled) into double-stranded DNA blocks. The double-stranded blocks are then subjected to one or more rounds of error correction or error elimination. Error correction/error elimination may include enzymatic catalysis-based error correction/error elimination and/or physical depletion-based error correction/error elimination. The corrected blocks are then subjected to polymerase chain reaction (PCR) to assembly the DNA template (e.g., gene) The DNA template may be up to 3-4 kilobases or more. The DNA synthesis and assembly module 12 is further configured to circularize the full-length DNA template as indicated by reference numeral 24. In particular, the DNA template is inserted into a vector to form a circular DNA template. In certain cases the DNA is circularized on itself. In certain embodiments, the steps performed by the DNA synthesis and assembly module 12 are automated. The DNA synthesis and assembly module 12 is configured to optimally generate approximately 10 to 100 nanograms of circular DNA template as indicated by reference numeral 26, but could generate more or less than this.

In certain embodiments, in a first workflow 11 (e.g., a single construct, large output mass process), a first type of DNA amplification module 14 (AMP A) is utilized. In certain embodiments, in a second workflow 13 (e.g., multi-construct, lower output mass system), a second type of DNA amplification module 15 (AMP B) is utilized. In certain embodiments, the DNA synthesis and assembly module 12 is configured to provide the DNA template as an input “seed” to either DNA amplification modules 14 and 15. In certain embodiments, a pre-made DNA input may be directly provided as the input to either DNA amplification modules 14 and 15. In either case, prior to being input into either DNA amplification modules 14 and 15, the circular DNA template is, or can be, treated with an exonuclease to degrade non-circular DNA and improve the quality of starting template material. (e.g. removal of non-circularized template and/or background). In certain embodiments, treatment of the circular DNA with the exonuclease is conducted by the DNA synthesis and assembly module 12.

The DNA amplification module 14 is configured to generate (e.g., amplify) a DNA product (e.g., endotoxin free DNA product) from the circular DNA template utilizing rolling circle amplification (RCA). The DNA product (e.g., RCA product) is a high molecular weight concatemer and highly branched (e.g., hyperbranched). In certain embodiments, the rolling circle amplification occurs via utilizing two-stage amplification reactions under isothermal conditions. The DNA amplification module 14 includes a first stage bioreactor (e.g., reaction vessel) configured for performance of a first stage of two-stage amplification reactions and a second stage bioreactor configured for performance of a second stage of the two-stage amplification reactions. The first stage bioreactor is configured for a first rolling circle amplification reaction having a first volume and the second stage bioreactor is configured for a second rolling circle amplification reaction having a second volume that is greater than the first volume. For example, the first volume may be approximately 20 milliliters and the second volume may be at least approximately 2 liters. In certain embodiments, the DNA amplification module 14 may be utilized for in vitro transcription to generate an RNA product from the circular DNA template. In other embodiments, the rolling circle amplification reaction occurs in a microplate 17 under isothermal conditions when the DNA amplification module 15 is utilized for a low volume, high throughput process (e.g., for multiple molecules).

The DNA amplification modules 14 and 15 are configured to store on-board and to utilize lyophilized reagents (e.g., dNTPs, hexamers, enzyme, etc.) for single-stage or two-stage amplifications reactions. The lyophilized reagents include excipients, carbohydrate to help stabilize the reagents (e.g., enzyme) during storage. In particular, the DNA amplification modules includes a hydration system or station configured to rehydrate the lyophilized reagents prior to use in the amplification reaction. In certain embodiments, the lyophilized reagents are part of a kit and are single-use consumable. The level of excipient utilized in making the lyophilized reagents (upon rehydration) is below a critical threshold for amplification efficiency for the amplification reaction (i.e., enables the amplification reaction to occur efficiently). In cases where the excipient negatively impacts the amplification reaction, the reagents may be lyophilized in a concentrated form so that upon rehydration of the lyophilized reagents, the final concentration of excipient will be below a threshold concentration at which the amplification reaction is impacted negatively (e.g. reaction slows or stops, product made is not high quality, etc.). When the DNA amplification module 15 is utilized for low-volume high throughput processes, the lyophilized reagents may be rehydrated in individual wells of a microplate 19 and then transferred to respective individual wells 17 having DNA templates where parallel amplification reactions occur in respective individual wells of the multi-well plate 17.

The DNA amplification module 14 is also configured to perform real time inline monitoring (e.g., measurements and analytics) of the amplification reaction via a plurality of sensors (e.g., as part of a panel of sensors) to ensure the quality and quantity of the generated DNA product. One or more of the sensors may be configured to directly contact contents of the amplification reaction. The monitoring enables tracking of progress of the reaction and analyzing product quality and/or product quantity. The sensors enable the measurement of fluid flow rate, a differential pressure (and, thus, computed viscosity), mass, volume, pH, light scattering (via OD600), refractive index, and absorbance at 260 nanometers or other wavelengths. In certain embodiments, the sensors are part of a kit and are single-use consumable. In certain embodiments, the feedback from the sensors may be provided to a controller that controls operations of the DNA amplification module 14 (e.g., amplification reaction). In certain embodiments, the controller may cause the DNA product to be released to the purification module 16 upon a particular parameter (e.g. viscosity, via pressure and flow rate) falling within a defined range. In other embodiments, the controller may cause the DNA product to be released to the purification module 16 upon a particular parameter (e.g. the absorbance at 260 nm dropping to a defined range as a result of the dNTP being converted into DNA). Similar, quality control monitoring may occur when utilizing the DNA amplification module 15 in the workflow 13.

As depicted in FIG. 1, the DNA amplification module 14 hydrates the reagents utilizing a hydration system as indicated by reference numeral 28. The circular DNA template is mixed in with the rehydrated reagents as indicated by reference numeral 30. The mixing may occur within a reaction vessel, the rehydration vessel, or a different vessel. Isothermal conditions are held while the amplification reaction proceeds as indicated by reference numeral 32. As noted above, performance of the first stage of two-stage amplification reactions in a first stage bioreactor at a first lower volume (e.g., approximately 20 milliliters). An amount of DNA product generated by the first-stage minimally ranges between 10 to 100 milligrams as indicated by reference numeral 34.

The reaction is optionally transferred to a second stage bioreactor for the second stage of the two-stage amplification reactions. For the second stage, the volume of the reaction is increased (e.g., to at least approximately 2 liters) and the process is repeated as indicated by reference numeral 36. An amount of DNA product outputted from the second stage of the two-stage amplification reactions minimally ranges between 100 to 2000 milligrams as indicated by reference numeral 38. During each stage of the two-stage amplification reactions the reactions (e.g., reaction conditions and kinetics) as well as the generated DNA product are being monitored as indicated by reference numeral 40. In the first workflow 11, the DNA amplification module 14 provides the generated DNA product to the purification module 16.

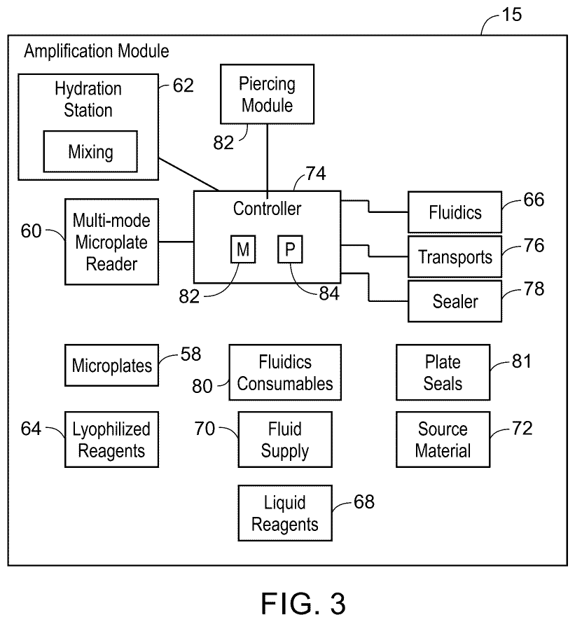

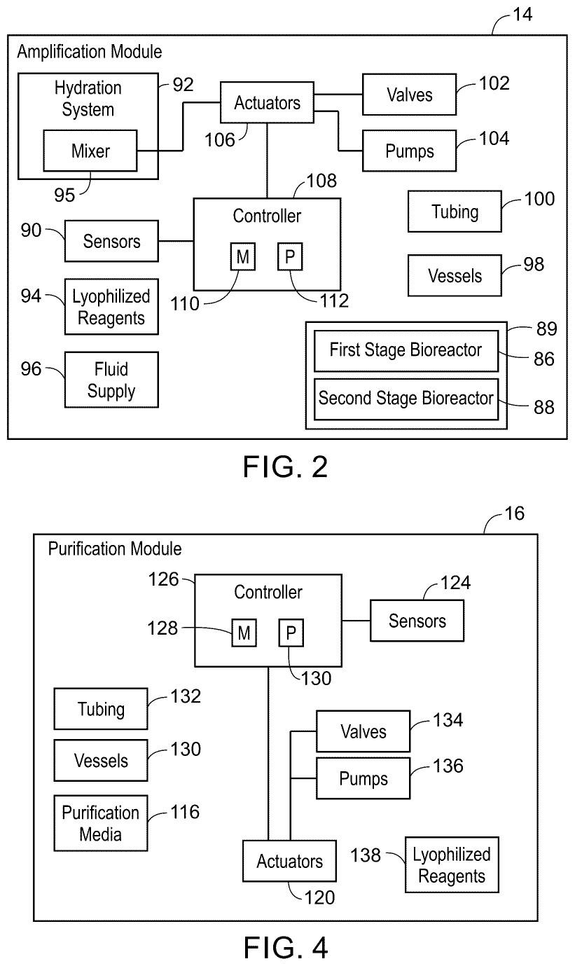

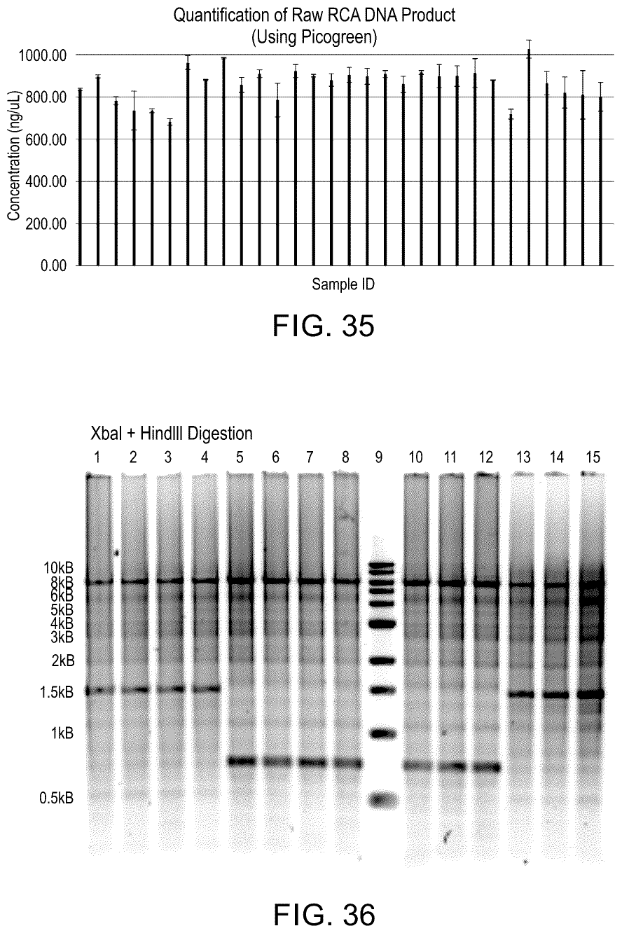

Similarly, the DNA amplification module 15 hydrates the reagents in individual wells of plate 19 utilizing a hydration system. The DNA amplification module 15 then transfers the rehydrated reagents to individual wells (e.g., optionally containing different DNA molecules or constructs) of the multi-well plate 17. Isothermal conditions are held while the amplification reactions proceed in the individual wells (e.g., for up to 8 hours). Reaction volumes in the individual wells may be up to 500 microliters in a 96-well plate format. Each reaction in reach respective well may minimally yield between 300 and 500 micrograms of DNA product. FIG. 35 depicts the yield of DNA products (e.g., from rolling circle amplification reactions) from multiple constructs utilizing the DNA amplification module 15 in FIG. 2. Each amplification reaction occurred in a separate well of the plate with a different DNA construct or template. Offline quantification was conducted utilizing Quant-iT™ PicoGreen™. FIG. 36 depicts the same DNA products (e.g., from rolling circle amplification reactions) from the multiple constructs generated utilizing the amplification module 15 in FIG. 2 after offline XbaI and HindIII digestion to confirm different identities by agarose gel electrophoresis.

The purification module 16 is configured to purify the DNA product by removing protein (e.g., polymerase such as phi29), cleaning up the DNA product, (e.g. removing e.g. dNTP, dNMP, hexamer), and changing the buffer as indicated by reference numerals 42, 44, and 46, respectively. The purification module 16 is configured to utilize one or more chromatographic techniques (e.g., non-ethanol-based precipitation techniques) to effectively purify the DNA product. In particular, the purification module 16 is configured to utilize specific ligands and pore sizes to purify the DNA product. In certain embodiments, the purification module 16 is configured to utilize polyanionic ligands (e.g., heparin) in conjunction with sodium dodecyl sulfate (SDS) and ethylenediaminetetraacetic acid (EDTA) to remove proteins. The inclusion of SDS into the sample prior to purification results in superior removal of DNA polymerase. In certain embodiments, the purification module 16 is configured to utilize pores greater than 3 microns in ion-exchange based concentration of the DNA product via positive selection.

The purification module 16 includes a plurality of sensors to monitor the purification of the DNA product. The sensors enable measurement of pressure, mass, volume, pH, conductivity, and absorbance at 260 nanometers, 280 nanometers, or other wavelength. The purification module 16 provides the purified DNA product to the fill-finish module 18. The system 10 includes a sequencer 47 to sequence a portion of the purified DNA product as indicated by reference numeral 48. The purification module 16 is configured to receive feedback from the plurality of sensors performing real time inline measurements of kinetics and conditions of purification. Control signals are provided based on the feedback to regulate the purification and isolate a purified product.

The fill-finish module 18 is configured to aliquot the dose volume having the DNA product as indicated by reference numeral 50. The fill-finish module 18 is also configured to sterilely fill and close each dose as indicated by reference numeral 52. The fill-finish module 18 is configured to generate greater than 100 doses (e.g., up to 1000 doses) as indicated by reference numeral 54. In certain embodiments, the fill-finish module 18 is configured to fill a single bag with multiple doses and then utilize a multi-dose dispenser to sterilely aliquot each dose.

The system 10 may include other components not shown in FIG. 1. For example, the system 10 may include respective controllers for the modules, control cabinets, media storage and processing equipment, size analyzer, vial storage system, via feed system. The system 10 may also include a human-machine interface.

FIG. 2 is a schematic diagram of the DNA amplification module 14. The DNA amplification module 14 includes a first stage bioreactor or reaction vessel 86 and a second stage bioreactor or reaction vessel 88 for the two-stage amplification reaction (e.g., two-stage rolling circle amplification reaction). The first stage bioreactor 86 and the second bioreactor 88 are fluidly coupled. The first stage bioreactor 86 is configured for a first reaction volume and a second stage bioreactor 88 is configured for a second reaction volume that is greater than the first reaction volume. For example, the reaction volume for first stage amplification reaction in the first stage bioreactor 86 is approximately 20 milliliters and the reaction volume for the second stage amplification reaction in the second stage bioreactor 88 is at least approximately 2 liters (e.g., up to 4 liters). The bioreactors 86, 88 are disposed within an incubation chamber 89. The incubation chamber 89 is configured to maintain an isothermal temperature during amplification reaction. The utilization of a first stage bioreactor 86 and a second stage bioreactor 88 is an exemplary embodiment. In certain embodiments, the amplification module 14 may include one or more amplification bioreactors. For example, depending on the starting mass and the desired output mass, the reaction may take place in a single vessel or occur with 3 stages or more of amplification.

The DNA amplification module 14 also includes a plurality of sensors 90. In certain embodiments, one or more of the sensors 90 are configured to enable real time inline monitoring (e.g., measurements and analytics) of the amplification reaction to ensure the quality and quantity of the generated DNA product. In the case of two-stage amplification reactions, each amplification stage is monitored by the sensors 90. The sensors 90 enable tracking progress of the reaction and analyzing product quality and/or product quantity. One or more of the sensors 90 may be configured to directly contact contents of the two-stage amplification reactions. The sensors 90 may be part of a panel. In certain embodiments, one or more of the sensors 90 may measure parameters of an amplification reaction volume during fluid circulation from a bioreactor (e.g., bioreactor 86) through sensors 90 and back to the same bioreactor. Reaction volumes may also be circulated between vessels (from hydration system 92, through sensors 90, to bioreactor 88). Some of the sensors 90 may include spectrophotometers, pressure sensors, pH sensors, temperature sensors, mass, volume, and other sensors (e.g., conductivity sensor, conductivity sensors (e.g., electrodes), etc.). The sensors 90 enable the measurement of a plurality of parameters including pressure (and, thus, computed viscosity), pH, light scattering (via OD600), temperature, refractive index, and absorbance at 260 nanometers or other wavelengths. In certain embodiments, the sensors 90 are part of a kit and are single-use consumable. In other embodiments, the sensors 90 may be integrated into a vessel (e.g., the bioreactor vessel 86).

The DNA amplification module 14 further includes a hydration system 92. The hydration system 92 is configured to rehydrate lyophilized reagents 94 (e.g., enzymes, hexamers, dNTPs, etc.) stored in a vessel. The lyophilized reagents 94 are stored mixed together as a single unit (e.g., cake) or as individual lyophilized reagents 94 stored as separate units (i.e., enzymes, hexamers, and dNTPs are all separate). The hydration system 92 is configured to provide a fluid (e.g., buffer or water for injection (WFI) water) from a fluid supply 96 to rehydrate lyophilized reagents 94. In certain embodiments, the rehydration may occur within the bioreactors (e.g., bioreactor 86). In certain embodiments, the DNA amplification module 14 include other vessels 98 (e.g., storage vessel(s) for lyophilized reagents 94 or intermediate mixing vessel). In certain embodiments, the hydration system 92 includes a mixer 95 for facilitating mixing between a fluid and the lyophilized reagents 94 in a vessel during rehydration.

The DNA amplification module 14 also includes tubing 100 to form fluid pathways for the hydration system 92, a sampling pathway, and fluid pathways between the bioreactors 86, 88 and other components (e.g., sensors 90) of the DNA amplification module 14. The DNA amplification module 14 includes a plurality of valves 102 (e.g., pinch valves) disposed along these pathways. The DNA amplification module 14 includes one or more pumps 104 (e.g., peristaltic pumps) to facilitate flow along the pathways. In certain embodiments, the vessels 98, tubing 100, and sensors 90 may be components of a single-use pre-sterilized pyrogen-free kit. These components interact with the valves, actuators, and pump such that fluid is not in direct contact with the devices used for fluid manipulation. In certain embodiments, the DNA amplification module 14 may utilize multiple use sterile connectors (e.g., such as INTACT™ connectors from Medinstill) to avoid any contact between a liquid path and an external environment.

The DNA amplification module 14 further includes a plurality of actuators 106. The actuators 106 may be associated with the valves 102, pumps 104, or other components of the DNA amplification module 14. The DNA amplification module 14 also includes a controller 108. The actuators 106, pumps 104, and sensors 90 are communicatively coupled to a controller 108. The controller 108 is configured to control the operations of the hydration system 92, the movement of fluid between vessels 98, bioreactors 89, within the tubing 100, through the sensors 90, from the fluid supply 96, and the conducting and monitoring of the amplification reactions. The controller 108 is also configured to receive feedback from the sensors 90 and to monitor the progress of the amplification reaction. In certain embodiments, the controller 108 may cause the DNA product to be released to the purification module 16 upon a particular parameter (e.g. viscosity (computed via pressure and flow rate) falling within a defined range). The controller 108 includes a memory 110 storing instructions and a processing system 112 to execute the instructions on the memory 110. As an example, the memory 110 may store processor-executable software code or instructions (e.g., firmware or software), which are tangibly stored on a non-transitory computer readable medium. Additionally or alternatively, the memory 110 may store data. As an example, the memory 110 may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, the processing system 112 may include multiple microprocessors, one or more “general-purpose” microprocessors, one or more special-purpose microprocessors, and/or one or more application specific integrated circuits (ASICS), or some combination thereof. For example, the processing system 112 may include one or more reduced instruction set (RISC) or complex instruction set (CISC) processors. The processing system 112 may include multiple processors, and/or the memory 110 may include multiple memory devices.

FIG. 3 is a schematic diagram of the DNA amplification module 15. The DNA amplification module 15 includes a plurality of microplates 56 that serve as a vessels for amplification reactions. The rolling circle amplification reaction occurs in a microplate 58 under isothermal conditions when the DNA amplification module 15 is utilized for a multi construct and low mass use case that requires a high throughput process. Each well of the microplate 56 serves as a bioreactor (e.g., reaction vessel) for a rolling circle amplification. The DNA amplification module 15 includes a multi-mode microplate reader 60 that contains a temperature control module (cooling and heating) for conducting the rolling circle amplification in the microplate 56 under isothermal conditions. The DNA amplification module 15 also utilizes the multi-mode microplate reader 60 to enable real-time at-line and online monitoring (e.g., measurements and analytic) of the amplification reaction to ensure the quality and quantity of the generated DNA product. In certain embodiments, the multi-mode microplate reader 60 may measure parameters of the reactions in the wells serving as bioreactors in the microplate 58. Some of the measurements may include absorbance, light scattering, volume, and fluorescence. The multi-mode microplate reader enables the measurement of a plurality of parameters including light scattering (OD500-OD900), refractive index, and absorbance at 260 nanometers and other wavelengths. In certain embodiments, the multi-model microplate plate reader 60 may directly measure the bioreactor wells on microplate 58 or sub-sample the bioreactor volume utilizing a secondary or plurality of microplates 58.

The DNA amplification module 15 further includes a hydration station 62. The hydration system 62 serves as a location to rehydrate lyophilized reagents 64 (e.g., enzymes, hexamers, dNTPs, etc.) stored in a vessel. The lyophilized reagents 64 are stored mixed together as a single unit (e.g., cake) or as individual lyophilized reagents 64 stored as separate units (i.e., enzymes, hexamers, and dNTPs are all separate). The hydration system 64 is configured to provide a fluid (e.g., buffer or water for injection (WFI) water) from a fluid supply 66 to rehydrate lyophilized reagents 64. In one embodiment, the rehydration may occur within the bioreactors within the wells on a microplate 58. In certain other embodiments, the DNA amplification module 15 may include other microplates 58 (e.g., storage vessel(s) for lyophilized reagents 64 or intermediate mixing vessel) that are used within the hydration station 62.

The DNA amplification module 15 utilizes fluidics 66 to move liquid reagents 68, fluid supply 70, lyophilized reagents 64, and source material 72 from one location to another across the microplates 58 on the system. The source material may either originate from the assembly module 12, as described earlier, or instead be a pre-made circular DNA input.

The DNA amplification module 15 also includes a controller 74. The fluidics 66, transports 76, multi-mode microplate reader 60 and sealer 78 are communicatively coupled to the controller 74. The controller 74 is configured to control the operations of the hydration station 62, the movement of fluid between fluidics consumables 80, microplates 58, lyophilized reagents 64, fluid supply 70, and source material 72, and the use of plate seals 881 with the sealer 78. The controller 74 also optionally controls the use of the piercing module 82 which may be used across a plurality of microplates 58 to effectively pierce plate seals 81 during liquid dispensing and/or aspiration during fluidics 66. The controller 74 is also configured to receive feedback from the multi-mode microplate reader 60 and to monitor the progress of the amplification reaction. In certain embodiments, the controller 74 may cause the DNA product to be released for purification upon reading a particular parameter (e.g. absorbance or light scattering falling within a defined range). The controller 74 includes a memory 83 storing instructions and a processing system 84 to execute the instructions on the memory 83. As an example, the memory K may store processor-executable software code or instructions (e.g., firmware or software), which are tangibly stored on a non-transitory computer readable medium. Additionally or alternatively, the memory 83 may store data. As an example, the memory 83 may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, the processing system 84 may include multiple microprocessors, one or more “general-purpose” microprocessors, one or more special-purpose microprocessors, and/or one or more application specific integrated circuits (ASICS), or some combination thereof. For example, the processing system 84 may include one or more reduced instruction set (RISC) or complex instruction set (CISC) processors. The processing system 84 may include multiple processors, and/or the memory K may include multiple memory devices.

The above embodiments of the single construct/large mass system (e.g., utilizing amplification module 14) and the multi-construct/lower mass system (e.g., utilizing amplification module 15) are not meant to be limiting. Some aspects of the single construct/large mass system may be applied to the multi-construct/lower mass system and vis-versa. Some components between the two types of systems may be common (e.g., utilization of lyophilized reagents, monitoring of reactions, etc.).

FIG. 4 is a schematic diagram of the purification module 16. The purification module 16 is configured to purify the DNA product generated by the DNA amplification module 14. In certain embodiments, the components of the purification module 16 may be similarly shared with the amplification module 14.

The purification module 16 utilizes one or more techniques to effectively remove protein and generate a protein-depleted DNA product. In particular, the purification module 16 includes purification media 116 (e.g. ligands on solid supports in the form of resin slurries, packed columns, or affinity membranes, or plates). In certain embodiments, the purification media 116 include affinity resins having ligands (e.g., heparin) for the depletion of proteins from the rolling circle amplification reactions. In certain embodiments, the purification media 116 include anion exchange materials with interstitial spaces of greater than 3 microns to enable purification of the protein-depleted DNA product without clogging. In preferred embodiments, affinity purification of the protein-depleted DNA product efficiently de-branches the rolling circle amplification reaction product by trapping hyperbranched species within the pores.

The purification module 16 includes a plurality of sensors 124 to monitor the purification of the DNA product. The sensors 124 enable the measurement of pressure (and, thus, viscosity), mass, volume, pH, conductivity, and absorbance at 260 nanometers, 280 nanometers, or other wavelengths.

The purification module 16 also includes a controller 126. The sensors 124 and one or more components of the purification module 16 are communicatively coupled to a controller 126. The controller 126 is configured to control the operations of the purification module 16 and the purification process. The controller 126 is also configured to receive feedback from the sensors 124 and to monitor the purification (e.g., quality) of the DNA product. The controller 126 includes a memory 128 storing instructions and a processing system 130 to execute the instructions on the memory 128. As an example, the memory 128 may store processor-executable software code or instructions (e.g., firmware or software), which are tangibly stored on a non-transitory computer readable medium. Additionally or alternatively, the memory 128 may store data. As an example, the memory 128 may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, the processing system 130 may include multiple microprocessors, one or more “general-purpose” microprocessors, one or more special-purpose microprocessors, and/or one or more application specific integrated circuits (ASICS), or some combination thereof. For example, the processing system 130 may include one or more reduced instruction set (RISC) or complex instruction set (CISC) processors. The processing system 130 may include multiple processors, and/or the memory 128 may include multiple memory devices.

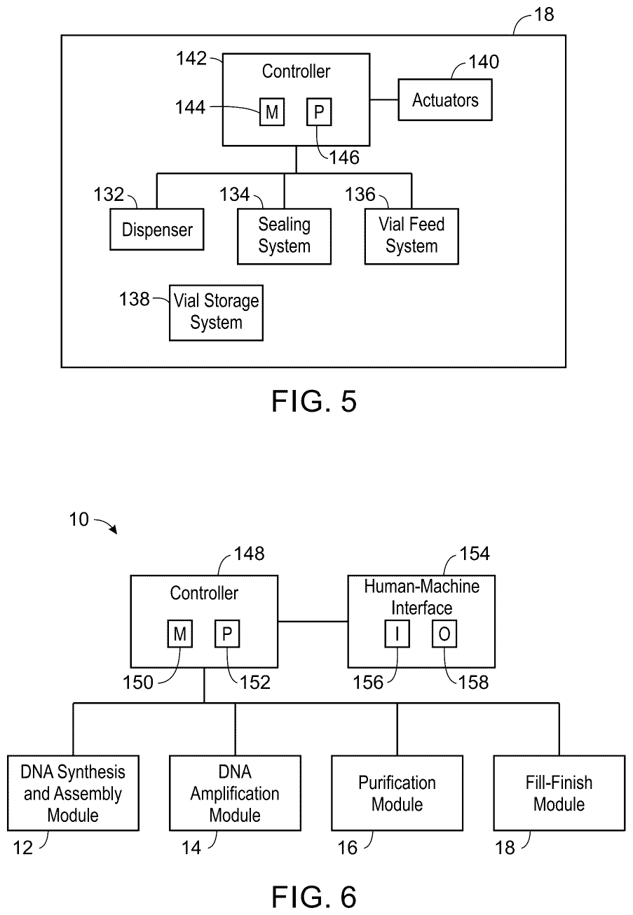

FIG. 5 is a schematic diagram of the fill-finish module 18. The fill-finish module 18 includes a fluid dispenser 132 configured to aliquot (in a sterile manner) a dose volume having the DNA product into a vessel. The vessel may be a standard glass vial, a plastic vessel, or a bag. The fill-finish module 18 also includes sealing system 134 to seal each vial upon aliquoting of the dose. In other embodiments, the vessel with the fluid aliquot may connect and disconnect to the filling system in manner that does not require a sealing step. The fill-finish module 18 further includes a vessel feed system 136 to provide vessel for the aliquoting. The fill-finish module 18 even further includes a vessel storage system 138 for storing the doses. The vessel may contain one or more doses. The fill-finish module 18 is also configured to sterilely fill and close each dose

The fill-finish module 18 includes actuators 140 associated with the components of the fill-finish module 18 or to move components within the fill-finish module 18. The fill-finish module 18 also includes a controller 142. The controller 142 is configured to control the operations of the fill-finish module 18. The controller 142 includes a memory 144 storing instructions and a processing system 146 to execute the instructions on the memory 144. As an example, the memory 144 may store processor-executable software code or instructions (e.g., firmware or software), which are tangibly stored on a non-transitory computer readable medium. Additionally or alternatively, the memory 144 may store data. As an example, the memory 144 may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, the processing system 146 may include multiple microprocessors, one or more “general-purpose” microprocessors, one or more special-purpose microprocessors, and/or one or more application specific integrated circuits (ASICS), or some combination thereof. For example, the processing system 130 may include one or more reduced instruction set (RISC) or complex instruction set (CISC) processors. The processing system 146 may include multiple processors, and/or the memory 144 may include multiple memory devices.

FIG. 6 is a schematic diagram of the system 10 in FIG. 1. In certain embodiments, the system 10 includes a controller 148 communicatively coupled to each of the DNA synthesis and assembly module 12, the DNA amplification module 14, the purification module 16, and the fill-finish module 18. In certain embodiments, the system 10 includes the controller 148 communicatively coupled to the DNA amplification module 15 instead of the DNA amplification module 14 (e.g., for amplification in a multi-construct/lower mass system). In particular, the controller 148 is communicatively coupled to the respective controllers of the modules. The controller 148 is configured to control the operations of the system 10. The controller 148 includes a memory 150 and a processing system 152. The memory 150 and the processing system 152 are similar to the memories and processing systems of the controllers of the respective modules. The system 10 includes a human-machine interface 154 communicatively coupled to the controller 148. The human-machine interface 154 enables a user to interface with the system 10. The human-machine interface 154 includes one or more input devices 156 (e.g., keyboard, microphone, touchscreen, mouse, etc.) and one or more output devices (e.g., display, speaker, etc.).

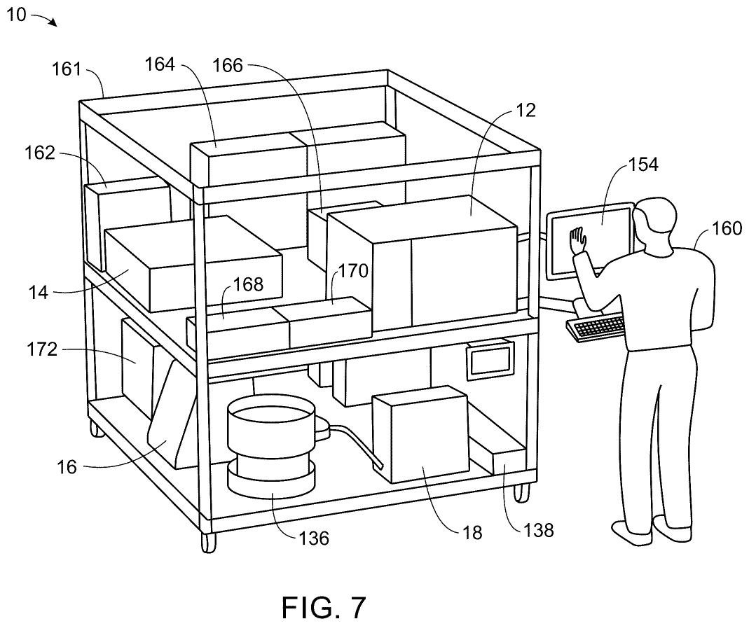

FIG. 7 is a schematic diagram of a user 160 interfacing with the system 10. As depicted, the system 10 is in a field deployable state. As depicted, the system 10 includes a mobile cart 161 with the components disposed of the system 10 disposed on it. The overall layout of the components of the system 10 and mode of transporting the system 10 may vary from that depicted in FIG. 7. As depicted, the user 160 interfaces with the system 10 via the human-machine interface 154. As depicted, the system 10 includes the DNA synthesis and assembly module 12, the DNA amplification module 14, the purification module 16, and the fill-finish module 18. In certain embodiment, the DNA amplification module 14 or purification module 16 may be associated with a transcription module to generate RNA. The system 10 also includes respective module controllers 162, control cabinets 164, media storage and processing 166. The system 10 further includes a size analyzer 168 (e.g., for performing automated DNA electrophoresis), a sequencer 170, a vial feed system 136, and a vial storage system 138. The system 10 may also contain a heater/chiller device 172 for maintaining a temperature-controlled environment for one or more of the modules. In other embodiments, the purification module 16 may generate the final product in a vessel or bag containing multiple doses that can be subsequently aliquoted into multiple doses.

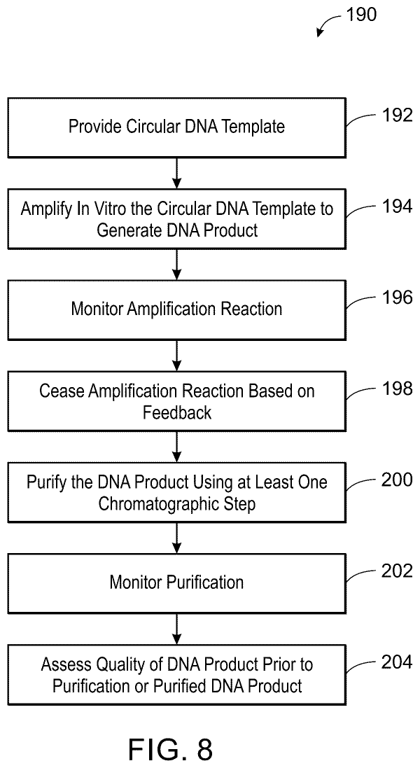

FIG. 8 is a flow chart of a method 190 for generating and purifying nucleic acid (e.g., DNA product). One or more steps of the method 190 may be performed by processing circuitry and modules of the system 10 in FIG. 1A and FIG. 1B (for utilization with workflow 11 or workflow 13 in FIG. 1). One or more steps of the method 190 may be performed simultaneously and/or in a different order from that shown in FIG. 8.

The method 190 includes providing a circular DNA template (e.g., single construct in an aqueous buffer) (block 192). In certain embodiments, the circular DNA template is provided directly from a DNA synthesis and assembly module to a DNA amplification module after generation of the circular DNA template. In certain embodiments, the circular DNA template is prepared externally from the system 10 and inputted into the DNA amplification module. Prior to providing the circular DNA template, the circular DNA template is pre-processed by treating it with an exonuclease to degrade non-circular DNA and improve the quality of the circular DNA template. In other embodiments, multiple DNA constructs (e.g., in respective aqueous buffers) may be provided (e.g., via liquid handling robot deck) from a DNA synthesis and assembly module to a DNA amplification module after generation of multiple circularized DNA templates. Prior to providing the circularized DNA templates, the circular DNA templates are pre-processed by treating them with an exonuclease to degrade non-circular DNA and improve the quality of the circularized DNA templates.

The method 190 also includes amplifying in vitro the circular DNA template to generate a DNA product (e.g., RCA product) (block 194). For example, the amplification may occur via a random-primed rolling circle amplification reaction under isothermal conditions. In certain embodiments, two-stage amplification reactions are utilized with a first rolling circle amplification reaction occurring in a smaller reaction volume in a first stage bioreactor and a second (subsequent) rolling circle amplification reaction occurring in a larger reaction volume in a second stage bioreactor. In certain embodiments, the rolling circle amplification reaction may occur at a temperature between 15 and 45 degrees Celsius. In certain embodiments, the rolling circle amplification reactions occurs at 30 degrees Celsius. In certain embodiments, the rolling circle amplification reactions occurs at 20 degrees Celsius. In other embodiments, for multiple circularized DNA templates, the rolling circle amplification occurs in vitro to generate respective DNA products (RCA products) from the circularized DNA templates. For example, the respective amplification reactions with the multiple DNA circularized DNA templates occur in respective wells of a multi-well plate in a low volume, high throughput process.

The method 190 further includes monitoring the amplification reaction by performing real-time measurements and analysis of reaction conditions and kinetics of the amplification reaction (block 196). The monitoring is performed by one or more sensors. In certain embodiments, the sensors are part of a panel of sensors. In certain embodiments, the monitoring is performed in-line. In certain embodiments, the sensor directly measure the reaction in the bioreactor. In certain embodiments, a portion of reaction volume is circulated and provided to the one or more sensors for analysis. In certain embodiments, the sensor directly measure the reactions in the microtiter plate. The method 190 includes ceasing the amplification reaction based on feedback from the monitoring in block 196 (block 198). For example, based on one or more measured parameters reaching a threshold or falling within a threshold range the reaction is ceased. Realtime inline monitoring of the purification process may be performed via a plurality of sensors in communication with a controller having a memory and a processor, wherein the controller is configured to receive feedback from the plurality of sensors and to control the purification process based on sensor feedback.

The method 190 even further includes purifying the DNA product utilizing at least one chromatographic step to obtain a purified DNA product (block 200). In certain embodiments, the at least one chromatographic step does not utilize alcohol. In certain embodiments, two chromatographic steps are utilized (e.g., a first selection utilizing a chromatographic resin having an affinity ligand to remove protein, followed by a second selection for the DNA product utilizing an anion exchange material having a pore size of 3 microns or greater). The method 190 still further includes monitoring the purification of the DNA product (block 202). The monitoring is performed by one or more sensors. In certain embodiments, the sensors are part of a panel of sensors. In an ideal embodiment, the second purification process is configured to cause elution of substantially de-branched DNA. Branched DNA in this case is the product of a rolling circle amplification reaction in which the DNA product contains one or more branched structures, such as three-way junctions, or Y-shaped structures or four-way Holliday or cruciform structures resulting from replication forks or hybridization events that were not resolved during the amplification reaction. The presence or absence of branched DNA produced from an RCA reaction is defined by the migration pattern produced under standard agarose gel electrophoresis. Branched DNA remains in the sample loading well, whereas de-branched DNA is able to migrate into the gel as a function of molecular weight and electrical field.

The method 190 further includes assessing a quality of either the DNA product prior to purification or after purification by respectively determining a viscosity for a sample of either the DNA product prior to purification or the purified DNA product or both (block 204). In certain embodiments, assessing the quality of the DNA product occurs prior to purification (e.g., utilizing the feedback of the monitoring in block 196). In certain embodiments, it is the quality of the purified DNA product that is assessed (e.g., utilizing the feedback of the monitoring in block 202). In certain embodiments, assessing the quality of the purified DNA product includes determining an identity of the purified DNA product by digesting the purified DNA product with an endonuclease and then determining a size of the purified DNA product upon digestion by the endonuclease. In certain embodiments, assessing the quality of the purified DNA product includes determining a purity of the purified DNA product by determining if protein is present in the purified DNA product. In certain embodiments, assessing the quality includes determining a pH of a sample of either the DNA product prior to purification or the purified DNA product or both. In certain embodiments, assessing the quality of the purified DNA product includes sequencing the DNA product.

As noted above, the system 10 may store the reagents on-board the system. Reagents may include hexamers, dNTPs, and enzymes (e.g. phi29 DNA polymerase). In certain embodiments, the reagents are stored in a lyophilized form. For example, for preparation of shelf-stable lyophilized enzyme, an enzymatic reaction mixture may be combined with a mixture of carbohydrates (long and short-chain). This is then flash-frozen, usually by submersion on or indirect thermal contact with liquid nitrogen or in a freezer. The frozen sample is then moved into a −80 degree Celsius freezer and a vacuum is applied to slowly remove the water from the frozen sample by sublimation. During this process the carbohydrates “glassify”, encasing the included protein in a protective shell. The resulting solid structure can be stored at higher temperatures (albeit with protection from water/humidity) and can easily be rehydrated by simply adding water or buffer to reconstitute to the original volume. or greater than the original volume. In certain embodiments, the reagents may be lyophilized together as a single unit (e.g., as a cake). In certain embodiments, individual reagents may be lyophilized separately (i.e., as separate units). A hydration system is utilized to rehydrate the reagents for the reaction (e.g., rolling circle amplification reaction).

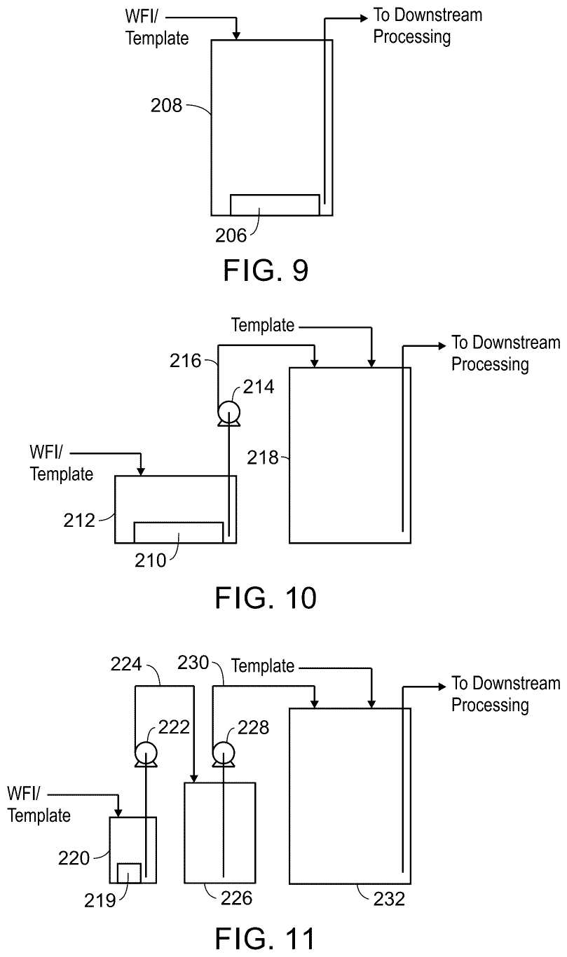

FIGS. 9-13 depicts different examples for the storage, rehydration, and utilization of the lyophilized reagents. FIG. 9 is a schematic diagram of a first scenario for the storage, rehydration, and utilization of the lyophilized reagents. As depicted, all of the lyophilized reagents (e.g., for the rolling circle amplification reaction) are stored as a complete cake 206 in a molecular bioreactor or reaction vessel or microtiter plate 208. Either water for injection (WFI) or buffer (provided via the hydration system and template (e.g., circular DNA template) are provided to the reaction vessel 208 where the reagents are hydrated and the reaction occurs. In certain embodiments, the template could be added before the WFI (or buffer), after the WFI (or buffer) is added, or combined with the WFI (or buffer). The reaction vessel 208 may be disposed on a mixing platform (e.g., nutator, orbital shaker, rocker, or similar) to promote mixing. A reaction may occur in the reaction vessel 208. The reaction product is then transferred to a downstream process. In certain embodiments, the reaction vessel 208 is configured for a larger volume (e.g., at least 20 milliliters). In certain embodiments, the reaction vessel 208 is an individual well of a plate.

FIG. 10 is a schematic diagram of a second scenario for the storage, rehydration, and utilization of the lyophilized reagents. As depicted, all of the lyophilized reagents (e.g., for the rolling circle amplification reaction) are stored as a complete cake 210 in a storage vessel 212. WFI or buffer (provided via the hydration system) and template (e.g., circular DNA template) are provided to the storage vessel 212 where the reagents are hydrated. In certain embodiments, the template is only provided into the reaction vessel 218. The storage vessel 212 may be disposed on a mixing platform to promote mixing. A pump 214 is then utilized to transfer the reaction mixture along a fluid pathway 216 from the storage vessel 212 to a molecular bioreactor or reaction vessel 218. Template may be provided to the reaction vessel 218. A reaction may occur in the reaction vessel 218. The reaction product is then transferred to a downstream process from the reaction vessel 218. In certain embodiments, the reaction vessel 218 is configured for a larger volume (e.g., at least 20 milliliters). In certain embodiments, the template could be added before the WFI (or buffer), after the WFI (or buffer) is added, or combined with the WFI (or buffer) when provided to the storage vessel 212. In certain embodiments, the template is provided directly to the reaction vessel 218. In certain embodiments, the reaction vessel 218 is an individual well of a plate. In certain embodiments, the storage vessel 212 is an individual well of a plate.

FIG. 11 is a schematic diagram of a third scenario for the storage, rehydration, and utilization of the lyophilized reagents. As depicted, all of the lyophilized reagents (e.g., for the rolling circle amplification reaction) are stored as a complete cake 219 in a storage vessel 220. WFI or buffer (provided via the hydration system) and template (e.g., circular DNA template) are provided to the storage vessel 220 where the reagents are hydrated. In certain embodiments, the template is only provided into the reaction vessel 232. A pump 222 is then utilized to transfer the reaction mixture along a fluid pathway 224 to an intermediate mixing vessel 226 for further mixing. The storage vessel 220 and/or the intermediate mixing vessel 226 may be disposed on a mixing platform to promote mixing. A pump 228 is then utilized to transfer the reaction mixture along a fluid pathway 230 from the intermediate mixing vessel 226 to a molecular bioreactor or reaction vessel 232. Template may be provided to the reaction vessel 232. A reaction may occur in the reaction vessel 232. The reaction product is then transferred to a downstream process from the reaction vessel 232. In certain embodiments, the reaction vessel 232 is configured for a larger volume (e.g., at least 20 milliliters). In certain embodiments, the template could be added before the WFI (or buffer), after the WFI (or buffer) is added, or combined with the WFI (or buffer) when provided to the storage vessel 220. In certain embodiments, the template is provided directly to the reaction vessel 232. In certain embodiments, the reaction vessel 232 is an individual well of a plate.

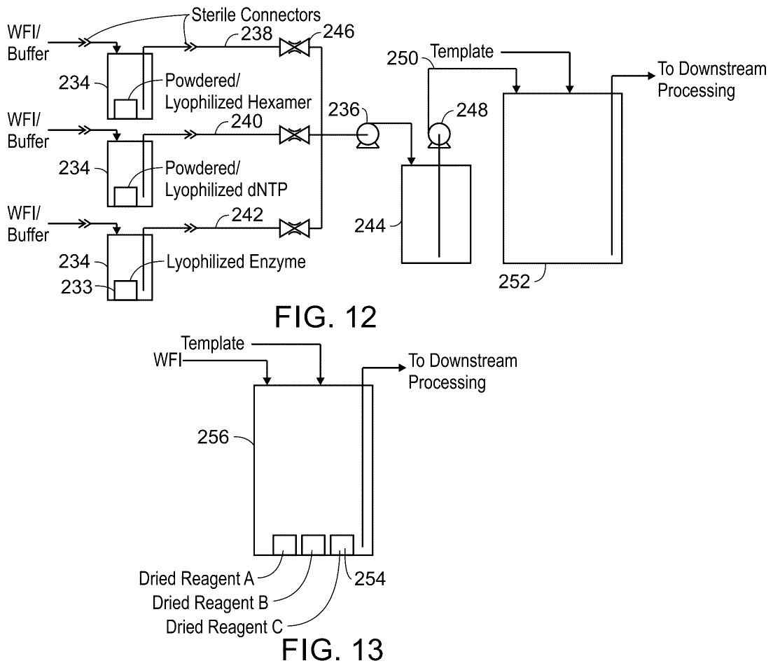

FIG. 12 is a schematic diagram of a fourth scenario for the storage, rehydration, and utilization of the lyophilized reagents. As depicted, individual lyophilized reagents (e.g., for the rolling circle amplification reaction) are stored separately as individual units 233 in separate storage vessels 234. WFI and/or buffer (provided via the hydration system) are provided to the storage vessels 234 where the reagents are hydrated. A pump 236 is then utilized to transfer the individually hydrated reagents along respective fluid pathways 238, 240, 242 to an intermediate mixing vessel 244 for further mixing. Valves 246 may be disposed along the respective fluid pathways to regulate the flow of the individual reagents to the intermediate mixing vessel 244 via pump 236. Sterile connectors 247 are disposed along fluid pathways into and out of the storage vessels 234. The storage vessels 234 and/or the intermediate mixing vessel 244 may be disposed on a mixing platform to promote mixing. A pump 248 is then utilized to transfer the hydrated reagent mixture along a fluid pathway 250 from the intermediate mixing vessel 244 to a molecular bioreactor or reaction vessel 252. Template (e.g., DNA circular template) is provided to the reaction vessel 252. A reaction may occur in the reaction vessel 252. The reaction product is then transferred to a downstream process from the reaction vessel 252. In certain embodiments, the reaction vessel 252 is configured for a larger volume (e.g., at least 20 milliliters). In certain embodiments, the reaction vessel 252 is an individual well of a plate. In certain embodiments, the intermediate mixing vessel 244 may be bypass such that the rehydrate individual reagents are directly pumped into the reaction vessel 252.

FIG. 13 is a schematic diagram of a fifth scenario for the storage, rehydration, and utilization of the lyophilized reagents. As depicted, individual lyophilized reagents (e.g., for the rolling circle amplification reaction) are stored as separate units 254 in a molecular bioreactor or reaction vessel 256. WFI or buffer (provided via the hydration system) and template (e.g., circular DNA template) are provided in the reaction vessel 256 where the reagents are hydrated and the reaction occurs. The reaction vessel 256 may be disposed on a mixing platform to promote mixing. A reaction may occur in the reaction vessel 256. The reaction product is then transferred to a downstream process. In certain embodiments, the template could be added before the WFI (or buffer), after the WFI (or buffer) is added, or combined with the WFI (or buffer) when provided to the reaction vessel 256. In certain embodiments, the reaction vessel 256 is configured for a larger volume (e.g., at least 20 milliliters). In certain embodiments, the reaction vessel 256 is an individual well of a plate.

Those familiar with the art can appreciate that these examples are not meant to be limiting. Other possible configurations/scenarios are possible. For example, template may be added to an intermediate mixing vessel and then contents transferred to the reaction vessel. In another example, template couple be added to the bioreactor vessel prior to the addition of the rehydrated reagents. In a further example, template could be combined with the WFI (or buffer). In certain embodiments, the rehydrated reagents are provided from one or more storage vessels or intermediate vessel to the reaction vessel where the template is either already present or added upon the rehydrated reagents being transferred to the reaction vessel.

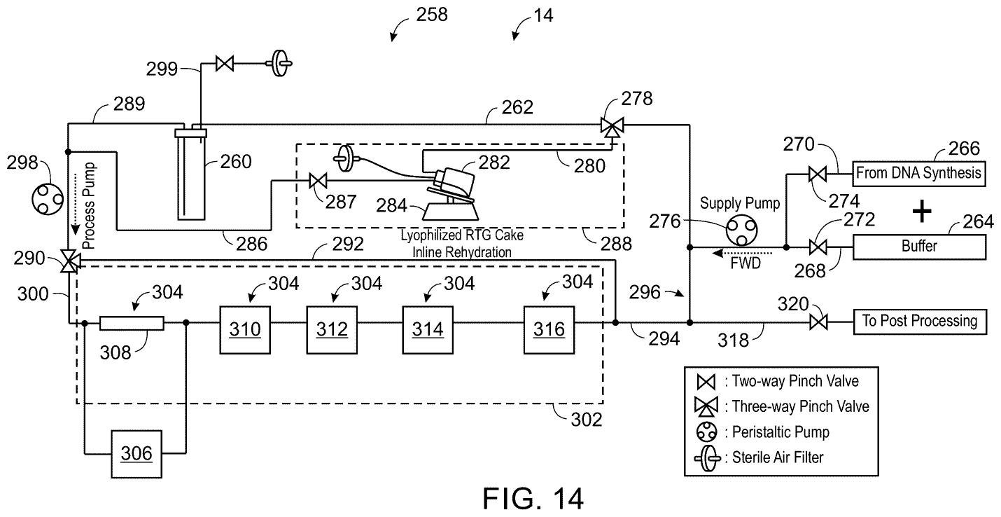

FIG. 14 is a schematic diagram of a fluid architecture 258 of an automated DNA amplification module 14 (e.g., for a large volume process for generating a single DNA product such as a vaccine). In certain embodiments, the fluid architecture 258 in FIG. 14 may be a first stage (e.g., small volume stage) of two-stage amplification reactions (e.g., rolling circle amplification reactions) that has a second subsequent stage (e.g., large volume stage). In certain embodiments, the fluid architecture 258 may vary from that depicted in FIG. 14. The DNA amplification module 14 includes a reaction vessel 260 for a small-scale reaction (e.g., 20 milliliters). The reaction vessel 260 is fluidly coupled via a fluid pathway 262 to a buffer supply 264 (e.g., WFI water or buffer) and DNA template source 266 (e.g., from DNA synthesis and assembly module 12 in FIG. 1). The buffer supply 264 is fluidly coupled to the fluid pathway 262 via fluid pathway 268. The DNA template source 266 is fluidly coupled to fluid pathway 262 via fluid pathway 270 coupled to the fluid pathway 268. Valves 272, 274 (e.g., two-way pinch valves) are respectively disposed along the fluid pathways 268, 270 to regulate flow of the buffer and the DNA template respectively. A supply pump 276 (e.g., supply channel of peristaltic pump) promotes the flow of the buffer and/or the DNA template along fluid pathway 268 to and along fluid pathway 262 towards the reaction vessel 260.

A valve 278 (e.g., three-way pinch valve) is disposed along the fluid pathway 262 to divert the buffer and/or the DNA template along fluid pathway 280 to a storage vessel 282 having a lyophilized reagent cake for the reaction stored within. The storage vessel 282 is disposed on a mixing platform 284 to promote rehydration of the reagents and mixing of the reaction. The storage vessel 282 is fluidly coupled to a fluid pathway 286. The fluid pathways 280, 286, the storage vessel 282, and the mixing platform 284 form an inline rehydration system 288. A valve 287 is disposed along the fluid pathway 286. The fluid pathway 286 is coupled to fluid pathway 289.

The fluid pathway 289 extends between the reaction vessel 260 and a valve 290 (e.g., three-way pinch valve). The valve 290 is also coupled to fluid pathway 292 which is coupled to fluid pathway 294. Fluid pathways 262, 289, 292, and 294 form a circulation loop 296 into and out of the reaction vessel 260. Process pump 298 (e.g., process channel of peristaltic pump) promotes flow along the circulation loop 296. The reaction volume is continuously circulated along the circulation loop 296. The process pump 298 also promotes flow along fluid pathway 286 from the hydration system 288 when the valve 287 is open. In certain embodiments, an air filter line 299 is coupled to the reaction vessel 260 to balance pressure in the reaction vessel 260 as fluid is transferred in and out.

The valve 290 is coupled to fluid pathway 300. An inline quality control panel 302 is disposed along the fluid pathway 300. The fluid pathway 300 is coupled to the fluid pathway 294. When the valve 290 is open, a portion of the reaction volume is diverted along the fluid pathway to flow through an inline quality control panel 302. The inline quality control panel 302 includes a plurality of sensors 304 to measure a number of parameters related to reaction kinetics and conditions. After flowing through the inline quality control panel 302, the portion of the reaction volume is provided back to the circulation loop 296. Those in the art would recognize that the valves could be stopcocks. Also, the three-way pinch valves could be made from two two-way pinch valves.

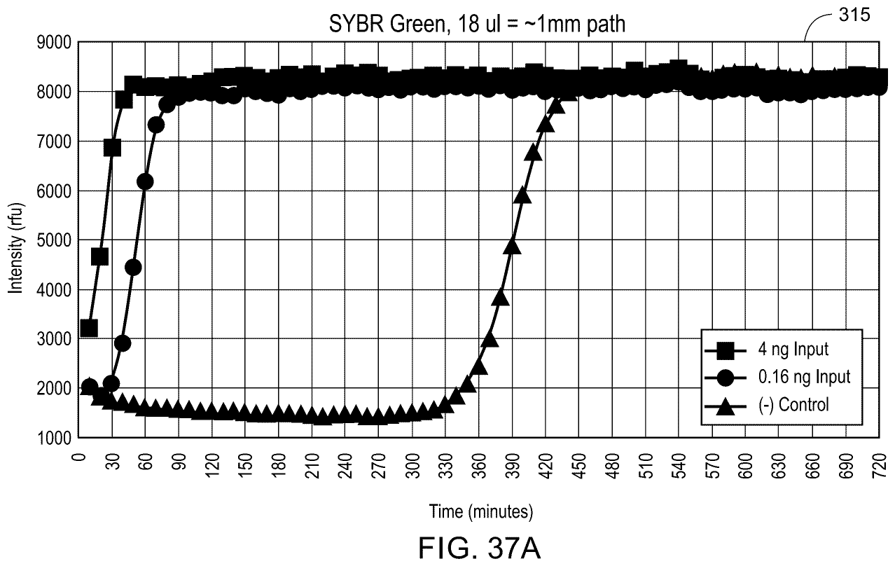

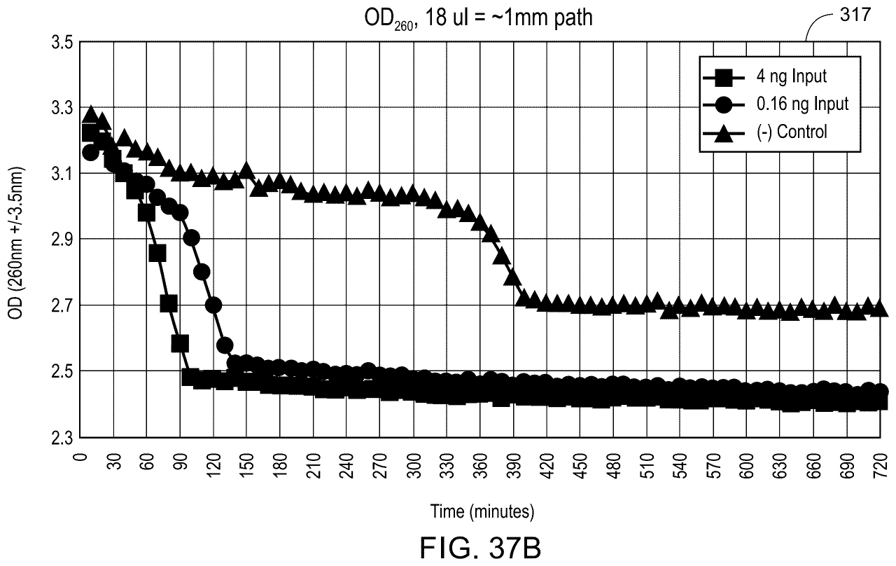

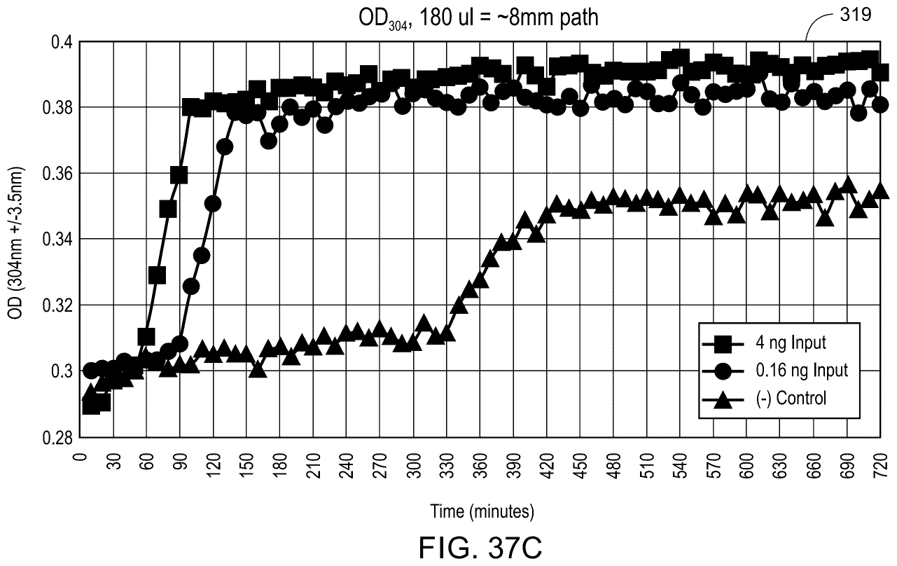

The sensors 304 include a pressure sensor 306 (e.g., differential pressure gauge). The pressure sensor 306 measures the differential pressure across an orifice 308 (e.g., fixed flow impedance or fluid impedance tube) disposed along the fluid pathway 300. The pressure drop across the orifice can be utilized to determine a viscosity of the portion of the reaction volume of the reaction. In certain embodiments, the differential pressure measurement can be measured by two discrete sensors on the inlet and outlet side of the orifice 308. The sensors 304 also include a temperature sensor 310 (e.g., temperature flow cell) to monitor a temperature of the portion of the reaction volume. The sensors 304 further include a pH sensor 312 (e.g., pH flow cell) to measure a pH of the portion of the reaction volume. In certain embodiments, the pH sensor 312 may include a pH optical chemosensor spot (e.g., within the pH flow cell) that directly contacts the portion of the reaction volume. The sensors 304 include an OD260 sensor 314 (e.g., quartz OD260 flow cell) to measure an optical absorbance of nucleic acids at 260 nanometers (nm) to determine a concentration of the nucleic acids. The OD260 sensor 314 has an optical pathway length of 0.2 mm. In certain embodiments, a cuvette having an optical pathway length of 0.2 mm may be utilized for the OD260 measurement. In certain embodiments, a cuvette having an optical pathway length of 0.1 mm may be utilized for the OD260 measurement. The path length utilized should be the minimal optical path length (i.e., minimal required to achieve a non-saturating absorbance). It has been surprisingly found that utilizing a short path length to measure the OD260 allows the sample to directly measured without dilution. Typical OD260 reading utilize 0.5 mm, 1 mm, or 10 mm path lengths, and at these path lengths DNA or RNA amplification reactions have too great a response to be accurately measured. By decreasing the path length being measured, it brings the OD260 of the sample down to levels that can be measured accurately. Additionally, owing to the difference in extinction coefficients at OD at 260 nm of nucleotides (0.0306) versus DNA (0.02) or RNA (0.025), the progress of DNA or RNA production can be monitored as the measured OD260 value decreases. Mass extinction coefficient is how strongly a substance absorbs light per mass density at a specific wavelength. It has surprisingly been found that while typical DNA monitoring utilizes the absorption of 260 nm light, which is the peak of absorption by DNA, RNA, and nucleotides, other wavelengths can be utilized. It has been found that while the OD at 260 nm of DNA and RNA synthesis reactions decreases as nucleotides are converted into product, owing to their distinct extinction coefficients at 260 nm, this reverses at higher wavelengths. At above approximately 292 nm, with a peak around 302-304 nm, where the absorption of this wavelength is over 10-fold less efficient than at 260 nm, there is an approximately 20 percent increase in absorption as nucleotides are converted into DNA or RNA product. This is quite useful, as it allows real time measurement of the reaction without having to decrease the path length to distances that can be difficult to move liquid through to avoid the absorption reading being off-scale. While utilizing a short path length (e.g. 0.2 mm) allows reactions having a large amount of nucleotide to be monitored using an OD at 260 nm and detecting product formation as a decrease in optical density, alternately in certain embodiments a longer path length (e.g. 10 mm) may be utilized while monitoring the OD at 304 nm, and product formation can be detected as an increase in optical density. This is illustrated in FIG. 37A-37C where real time monitoring of RCA reactions was performed in 96-well plates that allow for both fluorescent detection using an added intercalating dye, SYBR green (485 nm excitation, 535 nm emission), and optical absorption at either 260 nm or 304 nm. The various reactions were conducted in parallel, with a small volume of each in one well (creating a path length of approximately 1 mm), and a larger volume in another well (creating a path length of approximately 8 mm). In graph 315 of FIG. 37A, reactions were monitored for appearance of fluorescent signal caused by the DNA product binding SYBR-green dye which has been included in the reaction mixture. It can clearly be observed that the reaction with a larger amount of input template (4 ng), proceeds more rapidly than the reaction containing 0.16 ng of template, which in turn proceeded more rapidly than reactions containing no added template. This same trend is directly observed in graph 317 of FIG. 37B in which small-volume reactions (minimal path length) were monitored for OD at 260 nm, and again just as seen using SYBR green, it can clearly be observed by a decrease in OD that the reaction with a larger amount of input template (4 ng) proceeded more rapidly than the reaction containing 0.16 ng of template, which in turn proceeded more rapidly than reactions containing no added template. This demonstrates that OD at 260 nm can be utilized to monitor reactions directly if a short enough path length is used. However, by additionally including a larger reaction volume (graph 319 in FIG. 37C, ˜8 mm path length) where an OD at 304 nm was utilized, it can clearly be observed by an increase in OD that the reaction with a larger amount of input template (4 ng), proceeded more rapidly than the reaction containing 0.16 ng of template, which in turn proceeded more rapidly than reactions containing no added template. This demonstration shows that direct monitoring of DNA and RNA synthesis reactions containing high levels of nucleotide can be accomplished using a decrease in optical density when using a minimal path length and between 260 nm-285 nm light absorption, or utilizing a an increase in optical density at wavelengths above 292 nm, at approximately 304 nm (+/−10 nm) light absorption, or a combination. The overall goal of minimal path length optical density monitoring at wavelengths near the maximal absorption of nucleotides, DNA, and RNA, or monitoring of optical density where nucleotides, DNA, and RNA do not absorb efficiently, but also have different extinction coefficients, is the enablement of direct reaction monitoring. The sensors 304 also include an OD600 sensor 316 (e.g., OD600 flow cell). The OD600 sensor 316 measures an optical light scattering of the portion of the reaction volume at 600 nanometers. Sensor 316 could also measure optical absorbance of a portion of the reaction volume at other wavelengths (360 nanometers to 880 nanometers), or more specifically between 600 nanometers and 880 nanometers. This optical absorbance parameter enables detecting when the reaction ends. Pyrophosphates are produced as a byproduct of the rolling circle amplification reaction under certain buffer conditions. Once the pyrophosphate reaches a certain concentration it forms a magnesium salt and it precipitates out of solution indicating the completion of the reaction which can be detected using the OD600 sensor 316. The OD600 sensor 316 has an optical pathway length of 5 mm. The pH sensor 312 and OD260 sensor 314 can be utilized to determine a start of the amplification reaction, while each of the pH sensor 312, the OD260 sensor 314, and the OD600 sensor 316 can be utilized to determine the end of the amplification reaction. Determining viscosity based on the pressure differential measured by the pressure sensor 306 can also be utilized to determine the end of the amplification reaction. The measurements of the sensors 304 are not hindered by the excipients present in the reaction volume.

Those in the art would recognize that other sensors could be used. In addition, other absorbance and light scattering wavelengths could also be used. For example, a fluid flow rate sensor (non-contact ultrasonic based or in-line single-use) may be utilized. Fluid flow rate may be used in conjunction with the differential pressure measurement across a known orifice (i.e., length and diameter of a tube between the pressure sensors or pressure tap points) to compute viscosity and shear rate. Knowing viscosity and shear rate enables the system to compute specific properties of the polymers created during RCA as DNA is a shear thinning and thixotropic material.

Fluid pathway 318 is fluidly coupled to the circulation loop 296. Valve 320 (e.g., two-way pinch valve) is disposed along fluid pathway 318. Upon completion of the reaction, the valve 320 is opened to enable the reaction product to be sent for post-processing (e.g., purification by the purification module 16 in FIG. 1). The various valves and pumps are responsive to control signals (e.g., to actuators) from a controller. The various sensors provide feedback to the controller.

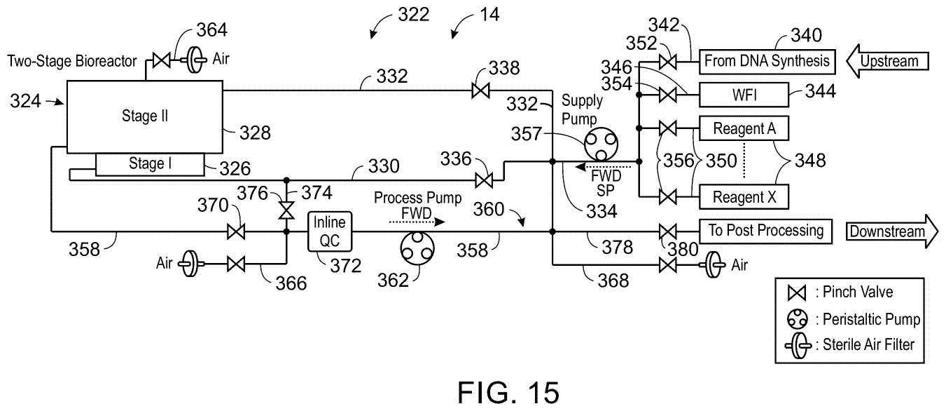

FIG. 15 is a schematic diagram of a fluid architecture 322 of an automated DNA amplification module 14 utilizing two-stage bioreactor 324 (e.g., for a large volume process for generating a single DNA product such as a vaccine). Although not shown, a hydration system may be utilized to rehydrate lyophilized reagents either in an inline manner or upstream of the bioreactor 324. The two-stage bioreactor 324 includes a first stage (e.g., small volume stage) reaction vessel 326 for a first rolling circle amplification reaction (e.g., having a reaction volume of 20 milliliters) and a second subsequent stage (e.g., large volume stage) reaction vessel 328 for a second rolling circle amplification reaction (e.g. having a reaction volume of 2 to 4 liters). The reaction vessel 326 is fluidly couple to the reaction vessel 328. In certain embodiments, the reaction vessel 326 is physically and directly coupled to the reaction vessel 328.

Both the reaction vessels 326, 328 are fluidly coupled via respective fluid pathways 330, 332 to fluid pathway 334. Valves 336, 338 (e.g., two-way pinch valves) are respectively disposed along the fluid pathways 330, 332 to regulate flow along them. A DNA template source 340 is fluidly coupled to the fluid pathway 334 via fluid pathway 342. WFI water supply 344 is fluidly coupled to the fluid pathway 334 via fluid pathway 346. One or more reagent supplies 348 are coupled to the fluid pathway 334 via respective fluid pathways 350. Valves 352, 354, 356 (e.g., two-way pinch valves) are respectively disposed along fluid pathways 342, 346 and 350 to regulate flow to the reaction vessels 326, 328 via the respective fluid pathways 332, 330 (via fluid pathway 334). A supply pump 357 (e.g., supply channel of peristaltic pump) promotes the flow of the DNA template, WFI water (or buffer), and reagents along fluid pathway 334 to and along fluid pathways 330, 332 towards the reaction vessels 326, 328.

The reaction vessel 328 is fluidly coupled to fluid pathway 358 which is coupled to the fluid pathway 332 Fluid pathways 358 and 332 form a circulation loop 360 into and out of the reaction vessel 328. Process pump 362 (e.g., process channel of peristaltic pump) promotes flow along the circulation loop 360. The reaction volume is continuously or discontinuously circulated along the circulation loop 360. In certain embodiments, an air filter line 364 is coupled to the reaction vessel 328 to balance pressure in the reaction vessel 328 as fluid is transferred in and out. Additional air filter lines 366, 368 may be disposed along the circulation loop 360.

A valve 370 (e.g., two-way pinch valve) is disposed along the fluid pathway 358. An inline quality control panel 372 is disposed along the fluid pathway 358. A fluid pathway 374 extends from a point between the valve 370 and the inline quality control panel 372. A valve 376 (e.g., two-way pinch valves) is disposed along the fluid pathway 374. A portion of the reaction volume flows through the an inline quality control panel 372. The inline quality control panel 372 includes a plurality of sensors to measure a number of parameters related to reaction kinetics and conditions as described above in FIG. 14. After flowing through the inline quality control panel 372, the portion of the reaction volume is provided back to the circulation loop 360.

Fluid pathway 378 is fluidly coupled to the circulation loop 360. Valve 380 (e.g., two-way pinch valve) is disposed along fluid pathway 378. Upon completion of the reaction, the valve 380 is opened to enable the reaction product to be sent for post-processing (e.g., purification by the purification module 16 in FIG. 1). The various valves and pumps are responsive to control signals (e.g., to actuators) from a controller. The various sensors provide feedback to the controller.

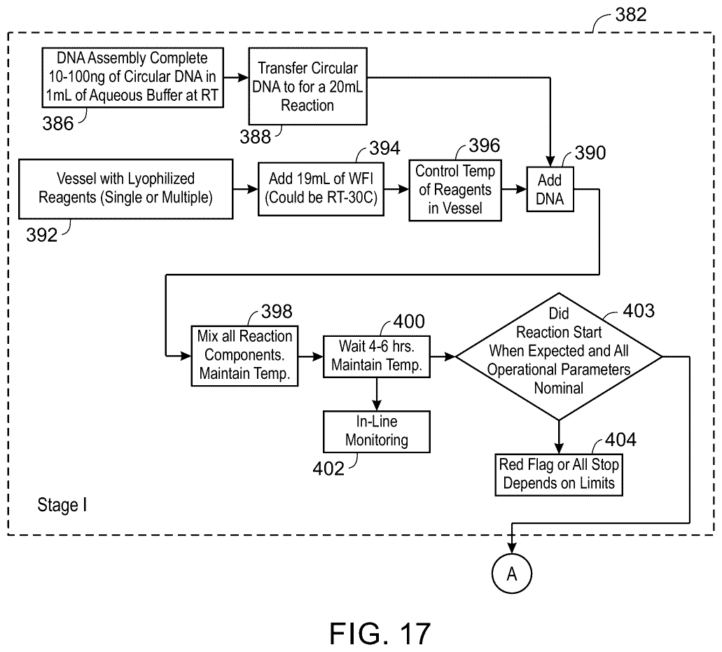

The reaction vessel utilized for amplification may vary. Examples of a multi-scale vessel include a 2-ply bag with ports on the bottom, a rigid vessel with a dip tube, a 2-ply bag with a funnel-like shape and ports at the bottom and top, a rigid vessel with a funnel-like shape with ports on bottom and top and/or with a dip tube, and a rigid vessel with a step shape (similar to bioreactor 324 shown in FIG. 17).

As an alternative to the fluid architectures 258, 322 for a large volume process for generating a single DNA product, a small volume, high throughput process is utilized. In the small, high throughput process wells within plates are utilized for the DNA amplification reactions.

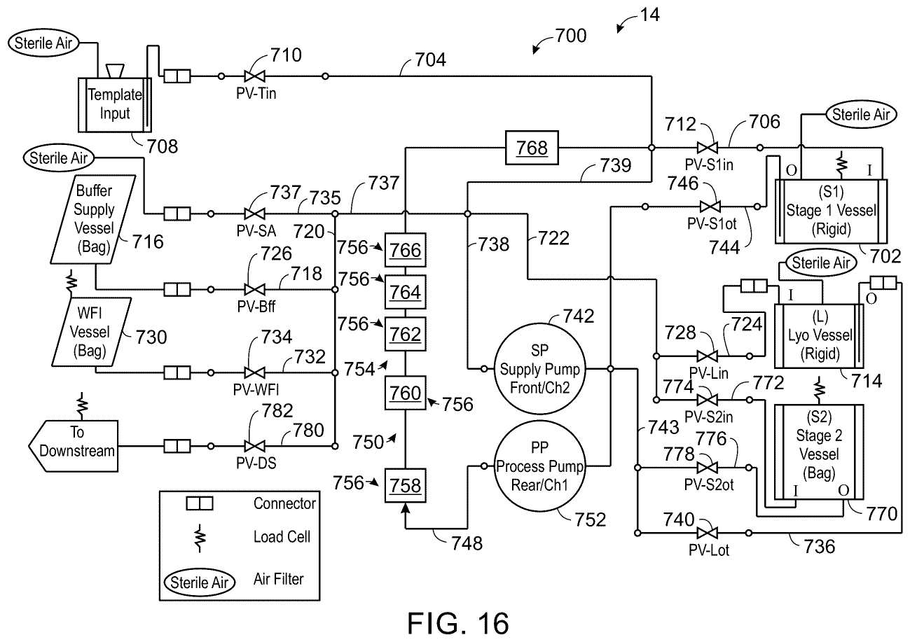

FIG. 16 is a schematic diagram of a fluid architecture 700 of an automated DNA amplification module 14 (e.g., for a large volume process for generating a single DNA product such as a vaccine). In certain embodiments, the fluid architecture 700 may vary from that depicted in FIG. 16. The fluid architecture 700 varies from the fluid architectures described in FIGS. 14 and 15 in a number of ways. For example, the stage 1 and stage 2 reaction vessels or bioreactors are connected to a fluid path. In addition, various vessels may be sterilely connected/disconnected to the main fluid path via connectors. Further, several of the vessels can be placed on respective load cells. Load cells can be used for monitoring volume in the vessels, but can also be used to estimate pumping volume rates into or out of a respective vessel. Similar to above, the measurement of the fluid rate via load cells can supplement or replace the direct fluid flow measurement when computing the viscosity and shear rate to determine the shear thinning and thixotropic nature of the DNA polymers formed during the RCA reaction.

The DNA amplification module 14 includes a first stage reaction vessel 702 (e.g., rigid vessel) for a small-scale reaction (e.g., amplification reaction). The first stage reaction vessel 702 is fluidly coupled via fluid pathways 744, 738, 739, 704, to a DNA template source 708. Valves 746, 710 (e.g., two-way pinch valves) are respectively disposed along the fluid pathways 744, 704 to regulate flow of the DNA template to the first stage reaction vessel 702. Supply pump 742 is disposed along fluid pathway 738 and promotes flow between the two vessels. In certain embodiments, the reaction buffer may be pumped from the first stage reaction vessel 702 to the template input vessel 708 prior to the addition of the template input material.

The DNA amplification module 14 also includes a lyophilized reagent vessel 714 (e.g., rigid vessel) fluidly coupled to a buffer supply vessel 716 (e.g., bag) via fluid pathways 736, 743, 738, 737, 720, 718. Valves 740, 734 (e.g., two-way pinch valves) are respectively disposed along fluid pathways 736, 724 to regulate flow of buffer to the lyophilized reagent vessel 714. Supply pump 742 is disposed along fluid pathway 738 and promotes flow between the two vessels. The DNA amplification module 14 includes a WFI vessel 730 (e.g., bag) fluidly coupled to the lyophilized reagent vessel 714 via fluid pathways 732, 720, 737, 738, 743, 736. Valves 734, 740 (e.g., two-way pinch valves) are respectively disposed along fluid pathways 732, 736 to regulate flow of WFI water to the lyophilized reagent vessel 714. Supply pump 742 is disposed along fluid pathway 738 and promotes flow between the two vessels. Buffer and/or WFI water may be provided to the lyophilized reagent vessel 714 to rehydrate the reagents.

The buffer supply vessel 716 is also fluidly coupled to the first stage reaction vessel 702 via fluid pathways 718, 737, 738, 744. Flow of buffer to the first stage reaction vessel 702 is regulated via valves 726 and 746. Supply pump 742 is disposed along fluid pathway 738 and promotes flow between the two vessels. The WFI vessel 716 is also fluidly coupled to the first stage reaction vessel 702 via fluid pathways 732, 720, 737, 738, 744 via supply pump 742. Flow of WFI water to the first stage reaction vessel 702 is regulated via valves 734 and 746. Supply pump 742 is disposed along fluid pathway 738 and promotes flow between the two vessels. Sterile air may be provided to various portions of the fluid architecture 700 via fluid pathway 735. Flow of the sterile air along the fluid pathway 735 is regulated via valve 737 (e.g., two-way pinch valve) and supply pump 742.

The lyophilized reagent vessel 714 is fluidly coupled to the first stage reaction vessel 702 via fluid pathways 736, 743, 738, 739, 706. Valves 740, 712 (e.g., two-way pinch valves) are respectively disposed along fluid pathways 736, 706 to regulate flow of the rehydrated reagents to the first stage reaction vessel 702. A pump 742 (e.g., supply pump) is disposed along fluid pathway 738 to promote flow. In certain embodiments, the DNA template may be provided to the lyophilized reagent vessel 714 instead of the first stage reaction vessel 702.

The reaction volume exits the first stage reaction vessel 702 via fluid pathway 744. Valve 746 (e.g., two-way pinch valve 746) and valve 712 are disposed along fluid pathways 744 and 706, respectively, to regulate flow of the reaction volume out of and into the first stage reaction vessel 702. Fluid pathways 744, 748, 706 form a circulation loop 750 into and out of the first stage reaction vessel 702. Process pump 752 is disposed along fluid pathway 748 and promotes flow along the circulation loop 750.

An inline quality control sensor panel 754 is disposed along the fluid pathway 748. When the valve 746 is open, a portion of the reaction volume is diverted along the fluid pathway to flow through the inline quality control panel 754. The inline quality control panel 754 includes a plurality of sensors 756 to measure a number of parameters related to reaction kinetics and conditions. After flowing through the inline quality control panel 754, the portion of the reaction volume is provided back to first stage reaction vessel 702. Those in the art would recognize that the valves could be stopcocks.

The sensors 756 include an OD260 sensor 758, an OD880 sensor 760, a temperature sensor 762, a pH sensor 764, a flow sensor 766, and a pressure sensor arrangement 768 as previously described. Those in the art would recognize that other sensors could be used. In addition, other optical absorbance/light scattering wavelengths could also be used. For example, a fluid flow rate sensor (non-contact ultrasonic-based or in-line single-use) may be utilized. Fluid flow rate may be used in conjunction with the differential pressure measurement across a known orifice (i.e., length and diameter of a tube between the pressure sensors or pressure tap points) to compute viscosity and shear rate. Knowing viscosity and shear rate enables the system to compute specific properties of the polymers created during RCA as DNA is shear thinning and thixotropic material.

Upon completion of the reaction in the first stage reaction vessel 702, the reaction volume may be transferred from the first stage reaction vessel 702 to a second stage reaction vessel 770 (e.g., bag) for a large scale reaction (e.g., amplification reaction). The first stage reaction vessel 702 is fluidly coupled to the second stage reaction vessel 770 via fluid pathways 744, 738, 722, 772. Valves 774, 746 regulate flow of the reaction product to the second stage reaction vessel 770. Supply pump 742 is disposed along fluid pathway 738 and promotes flow between the two vessels. Similar to first stage reaction vessel 702, buffer and/or WFI may be provided to the second stage reaction vessel 770 via the various fluid pathways and valves described above. Also, rehydrated reagents may be provided to the second stage reaction vessel 770 via the various fluid pathways and valves described above. Similarly to the first reaction, the second stage reaction may circulate through the inline quality control panel 754 via the various fluid pathways and valves as described above. In certain embodiments, the first stage or second stage reaction volume may be transferred from the reaction vessel to another vessel (for example, an emptied vessel 714) through the inline quality control panel 754 such that the change in mass of one of the vessel may be measured during the pump transfer such that pump flow rate can be calculated. In certain embodiments, the same lyophilized reagent vessel 714 used for the first stage reaction vessel 700 may be utilized. In certain embodiments, a different lyophilized reagent vessel to support a stage 2 reaction may be swapped out with the lyophilize reagent vessel 714 via the connectors. As sterile connect-disconnect components are used, the integrity of the rest of the kit is maintained. Similarly, different buffer bags may be utilized to support the stage reaction.

The reaction volume exits the second stage reaction vessel 770 via fluid pathway 776. Valve 778 (e.g., two-way pinch valve) is disposed along fluid pathway 776 to regulate flow of the reaction volume out of the second stage reaction vessel 770. Fluid pathway 780 is coupled to fluid pathways 720, 737, 738, 743, and 776. Valve 782 (e.g., two-way pinch valve) is disposed along fluid pathway 780. Upon completion of the second stage reaction, valve 782 and 778 are opened and the reaction product is transferred downstream (for post-processing, e.g., purification by the purification module 16 in FIG. 1) using the supply pump 742 disposed along fluid pathway 738 . . . . The various valves and pumps (e.g., actuators) are responsive to control signals from a controller. The various sensors provide feedback to the controller.

In this embodiment, ease-of-use and mistake proofing are increased over more traditional methods that utilize sterile connections (e.g., sterile tube fusion and sterile tube welding with multiple tubing tails). Those skilled in the art would appreciate that connectors could be added to the bioreactor vessels and or anywhere along the fluid path (e.g. around a sensor and around the entire quality control panel) to allow for easy exchanges of critical in-line components, eliminating the need to replace the entire kit for additional process steps, or if there is a bad sensor, etc.

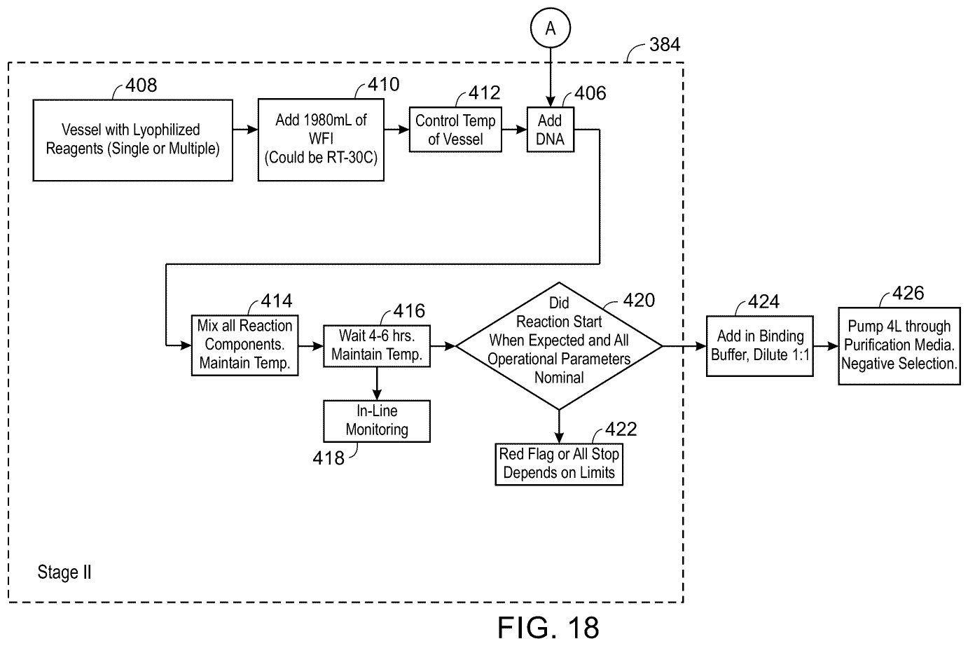

FIGS. 17 and 18 are schematic diagrams of a workflow for automated DNA amplification (e.g., rolling circle amplification with a two-stage bioreactor such as in FIG. 15). The workflow includes a first stage 382 and a second stage 384. In the first stage 382, upon completing the assembly of the DNA template, 10 to 100 nanograms of circular DNA template is obtained and is in 1 milliliter of aqueous buffer at room temperature (block 386). The circular DNA template is transferred to a reaction vessel (e.g., first stage reaction vessel) (block 388) where it is added (block 390) upon the reaction vessel reaching the desired temperature (block 396). It should be noted that the reaction may occur in more than one reaction vessel.

Also, in the first stage 382, in certain embodiments, one or more lyophilized reagents are present in the first stage reaction vessel (block 392). WFI, water-for-injection, or buffer (e.g., 19 milliliters) is added to the first stage reaction vessel to make a reaction volume of approximately 20 milliliters (block 394) once DNA template has been added (e.g. 1 milliliter). In certain embodiments, the reaction liquid added (block 394) may include amplification buffer components. In certain embodiments, the concentration of dNTPs in the first stage reaction is in the range of 0.4 millimolar to 1.6 millimolar. In certain embodiments, the air to liquid ratio is less than 100:1 and the pressure is maintained at 1 standard atmosphere (atm). The temperature of the first stage reaction vessel is adjusted to the desired temperature that is to be held during the reaction (block 396). All of the reaction components may be mixed while maintaining the desired temperature (block 398). The desired temperature is maintained for a set period of time (e.g., 4 to 8 hours) as the reaction occurs (block 400).

During the reaction under isothermal conditions, in-line monitoring of the reaction occurs (block 402). For examples, sensors as described in FIGS. 14 and 15 may be utilized for in-line monitoring of reaction kinetics and conditions. For example, OD260 may be directly measured. In certain embodiments, the reaction volume may be continuously circulated through sensors for in-line monitoring.

A determination is made as to if the reaction started when expected and if all operational parameters nominal (block 403). If the reaction did not start when expected or one or more parameters fall outside the nominal range or the reaction did not reach completion by a specific time, then a warning (e.g., red flag) may be provided and/or the reaction may be stopped depending on the circumstances (block 404). If the reaction did start when expected and all operational parameters are nominal and the reaction stopped as expected, the DNA reaction is provided to the second stage 384 (block 406).

Those in the art may recognize that the exact order of operations may vary slightly. In certain methods, the DNA may be added to the bioreactor before the reagents are added. In certain methods, the intermediate vessel may be used. Multi-sensor outputs may be combined to determine proper reaction kinetics throughout the entire reaction (i.e., start, middle, and end).

In the second stage 384, in certain embodiments, one or more lyophilized reagents are present in the second stage reaction vessel (block 408). WFI, water-for-injection, or buffer (e.g., 1980 milliliters) is added to the second stage reaction vessel to make a reaction volume of 2 liters (block 410) once DNA template from the first stage reaction vessel has been added (e.g. 20 milliliters). In certain embodiments, the reaction liquid added (block 410) may include amplification buffer components. In certain embodiments, the concentration of dNTPs in the second stage reaction for 2 liters is in the range of 0.4 millimolar to 1.6 millimolar. In certain embodiments, the reaction volume could be 4000 milliliters with the reaction at half the concentration. In certain embodiments, the air to liquid ratio is less than 100:1 and the pressure is maintained at 1 standard atmosphere (atm). The temperature of the second stage reaction vessel is adjusted to the desired temperature that is to be held during the reaction (block 412). In certain embodiments, the stage 2 reaction temperature may differ from the stage 1 reaction temperature. The reaction mixture from the first stage is added into the second reaction vessel (block 406). All of the reaction components may be mixed while maintaining the desired temperature (block 414). The desired temperature is maintained for a set period of time (e.g., 4 to 8 hours) as the reaction occurs (block 416). In certain embodiments, the stage 2 reaction time may differ from the stage 1 reaction time.

During the reaction under isothermal conditions, in-line monitoring of the reaction occurs (block 418). For examples, sensors as described in FIGS. 14 and 15 may be utilized for in-line monitoring of reaction kinetics and conditions. For example, OD260 may be directly measured. In certain embodiments, the reaction volume may be continuously circulated through sensors for in-line monitoring.

A determination is made as to if the reaction started when expected and if all operational parameters nominal (block 420). If the reaction did not start when expected or one or more parameters fall outside the nominal range or the reaction did not reach completion by a specific time, then a warning (e.g., red flag) may be provided and/or the reaction may be stopped depending on the circumstances (block 422). If the reaction did start when expected and all operational parameters are nominal and the reaction stopped as expected, the DNA reaction is subjected to purification (e.g., via the purification module 16 in FIG. 1) (blocks 424 and 426).

To purify the DNA product, a buffer is added to dilute the reaction volume in a 1:1 ratio (block 424) and facilitate optimal removal of protein from the DNA product.). The purification module then pumps the diluted reaction volume through a first purification process configured to remove protein and generate a protein-depleted DNA product. The protein-depleted DNA product is then pumped through a second to purification process configured to generate a purified DNA product. These purification steps are actuated and monitored using sensors that are configured along the fluid path. Purification is described in greater detail below.

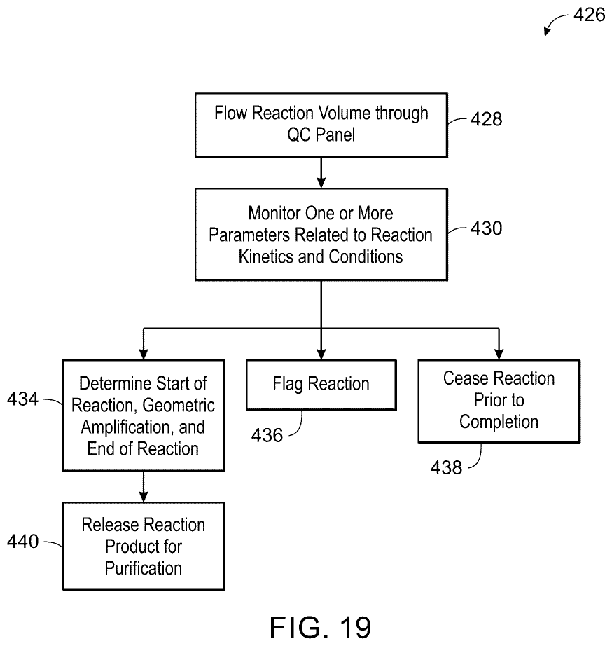

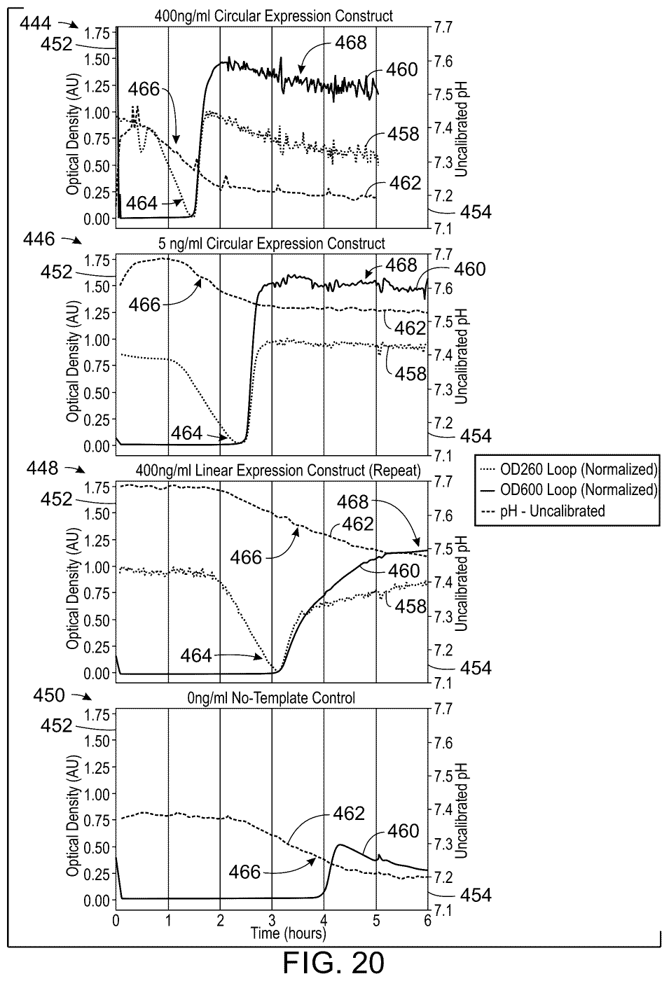

As mentioned above, the amplification reaction is monitored. FIG. 19 is a flow chart of a method 426 for monitoring a DNA amplification reaction. One or more steps of the method 426 may be performed by processing circuitry and the DNA amplification module of the system 10 in FIG. 1. One or more steps of the method 426 may be performed simultaneously and/or in a different order from that shown in FIG. 20.

In certain embodiments, the method 426 includes flowing a portion of an amplification reaction (e.g., rolling circle amplification reaction) through an inline quality control panel having a plurality of sensors (and then flowing the reaction volume back to the reaction vessel) (block 428). The plurality of sensors includes one or more of a pressure sensor (e.g., differential pressure gauge), a temperature sensor (e.g., temperature flow cell), a pH sensor (e.g., pH flow cell), an OD260 sensor (e.g., quartz OD260 flow cell), flow meter, and an OD600 sensor (e.g., OD600 flow cell). The pressure sensor measures the differential pressure across an orifice (e.g., fixed flow impedance or fluid impedance tube) disposed along a fluid pathway. The pressure drop across the orifice can be utilized to determine a viscosity of the reaction volume of the reaction. The viscosity can be correlated to DNA size and yield (e.g., computed product average molecular weight). The temperature sensor monitors a temperature of the reaction volume. The pH sensor measures a pH of the reaction volume. In certain embodiments, the pH sensor may include a pH spot (e.g., within the pH flow cell) that directly contacts the reaction volume. The OD260 sensor measures an optical absorbance of nucleic acids at 260 nm to determine a concentration of the nucleic acids. The flow meter measures the fluid flowrate through the quality control panel. In certain embodiments, the flow rate may be computed by measuring the change in weight of vessel in which the reaction volume is transferred into or out of. The measured flow rate can be utilized to determine the fluid shear rate across the orifice and used to determine the shear thinning and thixotropic behavior of the reaction fluid. The OD600 sensor measures an optical light scattering of the reaction volume at 600 nm. Those familiar in the art would recognize that light scattering can also be performed at alternative wavelengths, such as OD880.

The method 426 also includes monitoring one or more parameters related to reactions kinetics and conditions based on the feedback from the sensors (block 430). The method 426 includes 434 determining the start of the amplification reaction, geometric amplification, and end of the amplification reaction based on the feedback (block 434). The pH sensor and OD260 sensor can be utilized to determine a start of the amplification reaction, while each of the pH sensor, the OD260 sensor, and the OD600 sensor can be utilized to determine the end of the amplification reaction. Determining viscosity based on the pressure differential measured by the pressure sensor can also be utilized to determine the end of the rolling circle amplification reaction and characterize the product DNA (e.g. maintained in native concatemeric state). pH also provides an initial quality of the buffer. Further, initial OD260 value directly reflects the amount/concentration of dNTP in the starting reaction material. In certain embodiments (e.g., low volume, high throughput reactions), the parameters may be measured in the reaction vessel (e.g., one or more wells of a well plate).

In certain embodiments, the method 426 includes flagging the reaction (e.g., when one or more parameters fall outside a desired threshold range) (block 436). In certain embodiments, the method 426 includes ceasing or stopping the amplification reaction prior to completion (e.g., when one or more parameters fall outside a desired threshold range at a particular point (e.g., beginning of the reaction)) (block 438). In certain embodiments, if certain of the parameters fall within a respective threshold range at a certain point (e.g., at the end of the reaction), the reaction product is released for purification (block 440). For example, if viscosity falls within a desired threshold range, the reaction product may be released for purification or if the optical absorbance at 260 nanometer passes through a threshold value.

FIG. 20 depicts graphs 444, 446, 448, 450 of the monitoring of parameters during different rolling circular amplification reaction conditions. Graph 444 plots the monitored parameters for a rolling circular amplification reaction utilizing 400 ng/ml of a circular expression construct. Graph 446 plots the monitored parameters for a rolling circular amplification reaction utilizing 5 ng/mL of the circular expression construct. Graph 448 plots the monitored parameters for a rolling circular amplification reaction utilizing 400 ng/ml of a linear expression construct (PCR amplicon). Graph 450 plots the monitored parameters for a rolling circle amplification reaction without utilizing a template (i.e., no template control). Each graph 444, 446, 448, 450 includes a left y-axis 452 representing optical density (in absorbance AU units) and a right y-axis 454 representing uncalibrated pH. Each graph 444, 446, 448, 450 includes an x-axis 456 representing time (in hours). Plot 458 represents OD260 measurements (normalized) in the graphs 444, 446, 448. Plot 460 presents OD600 measurements (normalized) in the graphs 444, 446, 448, 450. Plot 462 represents pH (uncalibrated) in the graphs 444, 446, 448, 450.

A downward inflection (represented by reference numeral 464) in OD260 (i.e., plot 458) indicates the start of the amplification reaction. This indication occurs as nucleotides are being converted into DNA (or RNA). A downward inflection (represented by reference numeral 466) in pH (i.e., plot 462) also indicates the start of the amplification reaction. This indication occurs as H+ is generated during DNA or RNA synthesis. Saturation (represented by reference numeral 468) in OD600 (i.e., plot 460) indicates the end of the amplification reaction. This indication occurs as pyrophosphate is produced during DNA (or RNA) synthesis, and then complexes with Mg to produce an insoluble magnesium pyrophosphate salt. This salt then causes light scattering during optical analysis. It is not an optical density, but rather light scattering by the salt crystals.

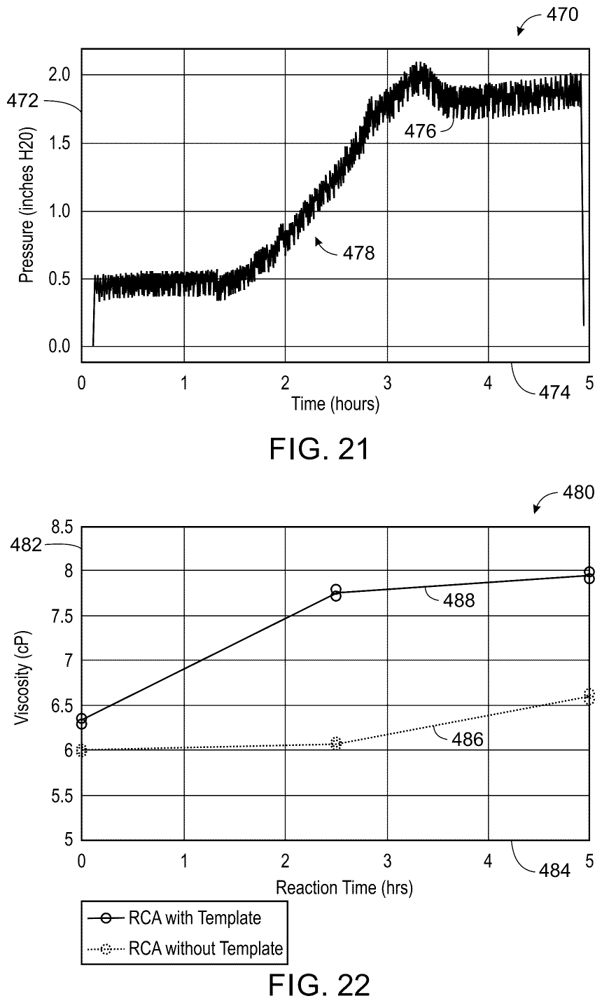

FIG. 21 depicts a graph 470 of monitoring of pressure during a rolling circle amplification reaction. The graph 470 includes a y-axis 472 representing pressure (in inches of a water column) and an x-axis 474 representing time (in hours). Plot 476 represents the measured pressure. An upward trend (represented by reference numeral 478) in pressure indicates the start of the amplification reaction.

FIG. 22 depicts a graph 480 of monitoring of viscosity at a given shear rate during a rolling circle amplification reaction. The graph 480 includes a y-axis 482 representing viscosity (in centipoise (cP)) and an x-axis 484 representing time (in hours). Plot 486 represents the measured viscosity for a rolling circle amplification reaction without template. Plot 488 represents the measured viscosity for a rolling circle amplification reaction with template at the same given shear rate. As depicted by plot 486, the viscosity steadily increase as the amplification reaction progresses. In certain embodiments, the viscosity is derived from differential pressure measurements. Once pressure is captured, and by knowing or measuring the flow rate imposed by a pump, viscosity (μ) can be calculated by the following equation (derived from Poiseuille's Law as in a laminar flow regime):

$$μ = \frac{Δ p {A}^{2}}{8 π L Q} \qquad ( 1 )$$

Where Q represents volumetric flow rate, Δp represents pressure drop, L represents length of a channel, and A represents a cross-sectional area. In certain embodiments, a viscometer may be utilized for measuring viscosity. In certain embodiments, the change in weight of a vessel containing the reaction volume as it is pumped through the channel may be used in conjunction with the density of the reaction mix to compute the flow rate. In certain embodiments, the computed viscosity may be transformed to a computed average molecular weight/size of polymers. It should be noted that the DNA product in the disclosed embodiments is not processed (i.e. digested, cleaved or sheared) before or after purification. Thus, the computed viscosity can be utilized as a quality metric of the product via the computed average molecular weight/size of the product DNA polymer. The DNA product in the disclosed embodiments is maintained as concatemeric repeat (polymer), so viscosity would be lost if the DNA polymer is processed enzymatically or physically into monomer units. In ideal embodiments, the DNA product is not treated with recombinases, endonucleases, shear forces, or other means to break the DNA concatemer.

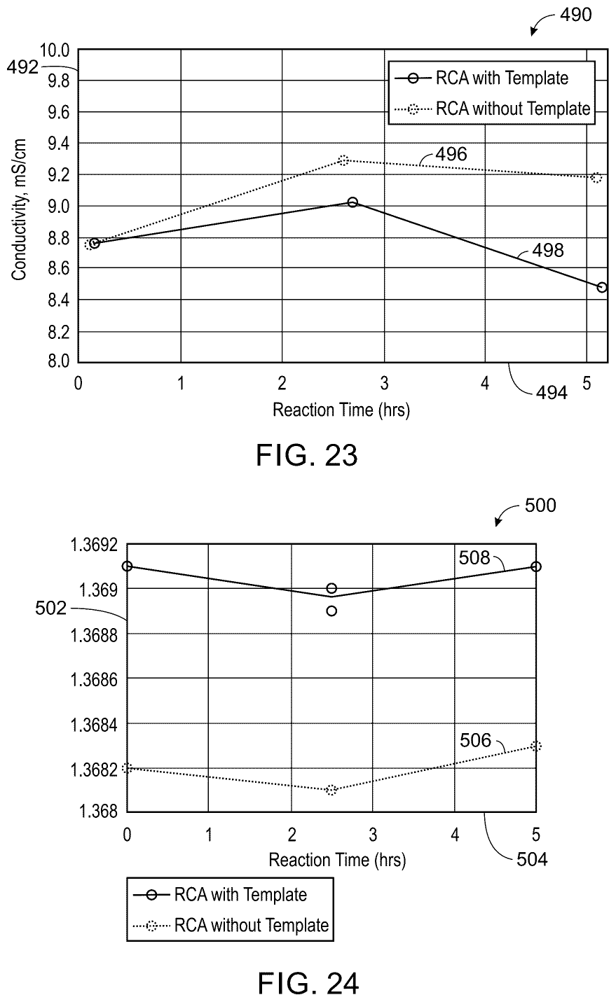

FIG. 23 depicts a graph 490 of monitoring of conductivity during a rolling circle amplification reaction. The graph 490 includes a y-axis 492 representing conductivity (in milliSiemens (mS) per centimeter (cm)) and an x-axis 494 representing time (in hours). Plot 496 represents the measured conductivity for a rolling circle amplification reaction without template. Plot 498 represents the measured conductivity for a rolling circle amplification reaction with template.

FIG. 24 depicts a graph 500 of monitoring of the refractive index during a rolling circle amplification reaction. The graph 500 includes a y-axis 502 representing refractive index (in milliSiemens (mS) per centimeter (cm)) and an x-axis 504 representing time (in hours). Plot 506 represents the measured refractive index for a rolling circle amplification reaction without template. Plot 508 represents the measured refractive index for a rolling circle amplification reaction with template.

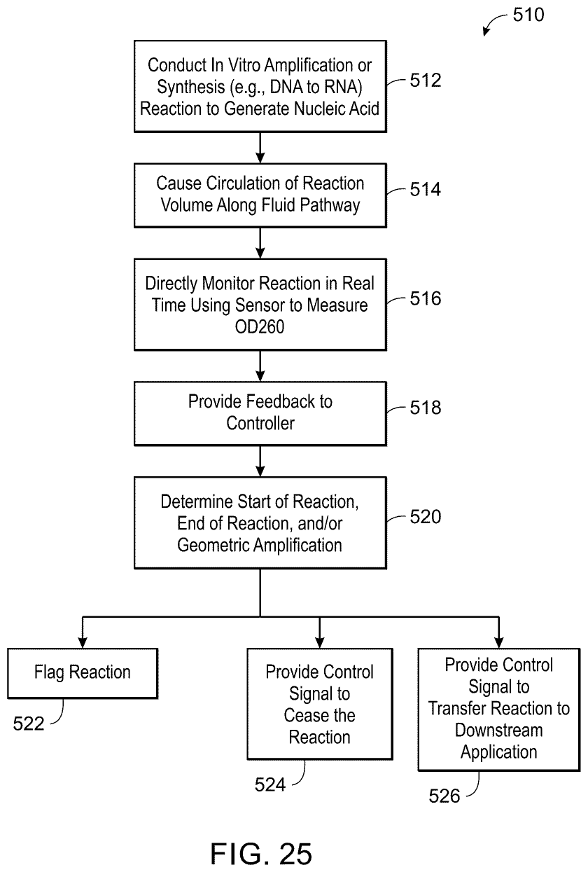

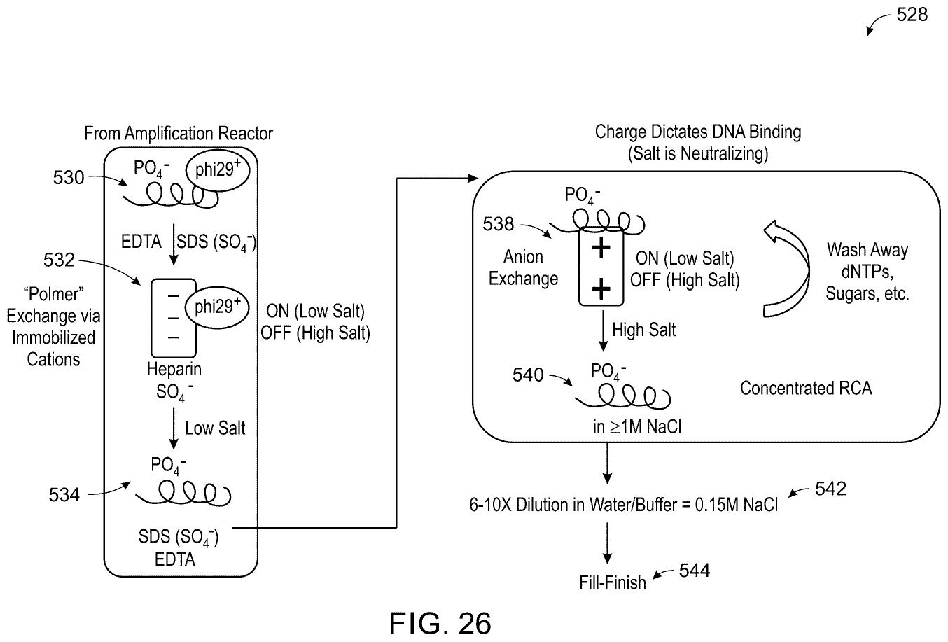

FIG. 25 is a flow chart of a method 510 for monitoring generation of a nucleic acid. One or more steps of the method 526 may be performed by processing circuitry and the DNA amplification module of the system 10 in FIG. 1A and FIG. 1B or another type of module for generating (e.g. amplifying or synthesizing) nucleic acid. One or more steps of the method 526 may be performed simultaneously and/or in a different order from that shown in FIG. 26.

The method 510 includes conducting in vitro an amplification reaction or a synthesis reaction (e.g., from DNA to RNA) to generate the nucleic acid (block 512). In certain embodiments, the nucleic acid may be DNA. In certain embodiments, the nucleic acid may be RNA. In certain embodiments, the reaction may be rolling circle amplification (e.g., for RNA or DNA). In certain embodiments, the reaction may be a transcription reaction. For example, control signals may be sent to equipment to conduct the reaction (e.g., modulate a temperature, add the reagents and the template together to generate the reaction volume, etc.).

In certain embodiments, the method 510 includes causing, via a pump, circulation of a reaction volume of the amplification reaction or the synthesis reaction along a fluid pathway to and from a reaction vessel. (block 514). For example, control signals may be sent to the pump to cause the circulation of the reaction volume. In other embodiments, the control signals may be sent to the pump to cause the fluid to shuttle back and forth between a reaction vessel and an intermediate vessel.

The method 510 further includes directly monitoring amplification reaction kinetics or synthesis reaction kinetics in real time utilizing a sensor configured to measure an absorbance at 260 nm (block 516). In certain embodiments, the absorbance may be measured between 230 and 300 nanometers. Utilizing the sensor measuring OD260 enables the detection of progress of a bulk or standard reaction under normal conditions and concentrations (i.e., without dilution). In certain embodiments, directly monitoring the amplification reaction kinetics or the synthesis reaction kinetics occurs without the utilization of dyes; this enables direct product interrogation without any added impurities or without utilizing anything that inhibits the reaction. Dye-based monitoring (e.g., dyes that only bind to double stranded DNA) is a conventional practice but downstream use of the amplified DNA is complicated by the bound dyes (which may require laborious denaturation/purification to remove these dyes). In certain embodiments, the sensor utilizes an optical path length of 0.2 millimeters to measure the absorbance at 260 nm. In certain embodiments, the measurement site for the sensor is disposed along the fluid pathway. In certain embodiments, the sensor comprises a flow cell (e.g., having an optical path length of 0.2 millimeters) integrated with the fluid pathway at the measurement site to enable continuous monitoring of variable volumes of the reaction volume (as it is being circulated) for nucleotide to nucleic acid conversion. This enables an inline system to support process analytical technology for manufacturing nucleic acid-based medical countermeasure products. In certain embodiments, the sensor is coupled to a cuvette e.g., having an optical path length of 0.2 millimeters) located at the measurement site. In certain embodiments, a cuvette having an optical pathway length of 0.1 mm may be utilized for the OD260 measurement. It has been surprisingly found that utilizing a short path length to measure the OD260 allows the sample to directly measured without dilution. Typical OD260 reading utilize 0.5 mm, 1 mm, or 10 mm path lengths, and at these path lengths typical DNA or RNA amplification reactions have too great a response (i.e. saturation or near-saturation) to be accurately measured. By decreasing the path length being measured, it brings the OD260 of the sample down to levels that can be measured accurately. Additionally, owing to the difference in extinction coefficients of nucleotides (0.0306) versus DNA (0.02) or RNA (0.025), the progress of DNA or RNA production can be monitored as the OD260 drops. Isothermal reactions are saturated with OD260 signal until a critical path length is reached. Although a 0.2 mm path length is mentioned, it should be noted that the minimum distance required to avoid saturation (determined empirically) may be utilized.

The method 510 even further includes providing feedback from the sensor to the controller (block 518). The method 510 still further includes determining (via the controller) a start of the amplification reaction or the synthesis reaction, an end of the amplification reaction or the synthesis reaction, and/or geometric amplification (when it is an amplification reaction) of the nucleic acid (block 520). In certain embodiments, the method 510 includes flagging (via the controller) the reaction when OD260 due to an abnormality based on the feedback (e.g., OD260 outside a range at a specific time or the kinetics of reaction not proceeding as expected) (block 522). In certain embodiments, the method 510 includes providing a control signal (via the controller) to cause the ceasing of the reaction prior to the end due to an abnormality based on the feedback (e.g., OD260 outside a range at a specific time or the kinetics of reaction not proceeding as expected) (block 524). In certain embodiments, the method 510 includes providing a control signal upon the reaction ending under normal conditions for the transfer of the reaction product to a downstream application (e.g., purification) (block 526). The monitoring ensures that the process proceeds as desired, thereby meeting quality metrics.

As noted above, in certain embodiments, the system utilizes a high volume to generate a single molecule (e.g., vaccine). Manufacturing of vaccine-grade DNA requires the removal of protein components that otherwise serve as antigens if preserved and co-administered with the DNA vaccine. FIG. 26 is a schematic diagram of a workflow 528 for purification of a DNA product (e.g., RCA product from RCA reaction). The workflow 528 enables the purification of the DNA product without utilizing alcohol (e.g., ethanol).

The workflow 528 includes obtaining the reaction volume having the DNA product (e.g., RCA product) as indicated by reference numeral 530 from the amplification reactor (e.g., of the DNA amplification module 14 in FIG. 1). The reaction volume is diluted in a buffer (e.g., at a ratio of 1:1) to facilitate optimal removal of protein from the DNA and form a diluted reaction volume. In certain embodiments, to facilitate the optimal removal of protein (e.g., polymerase such as phi29) from the diluted reaction volume, the buffer includes EDTA (e.g., at a final concentration of 40 millimolar) and SDS (e.g., at a final concentration of 0.2 percent). The addition of SDS is required to obtain effective removal of phi29 DNA polymerase. This may be due to SDS disrupting interactions between the polymerase and the DNA. The diluted reaction volume is inputted (e.g., for flow through) into a chromatographic vessel 532 having an affinity resin (e.g., immobilized heparin ligands) to select for protein in the diluted reaction volume, resulting in a protein-depleted DNA product (e.g., RCA product) in a low salt eluate 534 (e.g., less than 1 M sodium chloride). The chromatographic vessel 532 may be a column, non-packed resin bed in vessel, a moving bed system, or other type of vessel. The diluted reaction volume may alternatively be exposed to polyanionic ligand resin (e.g., immobilized cations) to select for protein in the diluted reaction volume using cation exchange where protein binds the resin and DNA does not. Removal of the resin after protein binding allows for removal of the protein from the unbound DNA. This affinity purification results in greater than 99 percent removal of protein including phi29 DNA polymerase. The resin matrix of the chromatographic vessel 532 may also be configured as a size exclusion matrix to remove small molecules such as SDS, which avoids concentrating the SDS during subsequent DNA purification steps (as SDS is also negatively charged like DNA). Removal of protein from an RCA reaction volume is depicted as set forth in Examples 1 and 2 below

Returning to the workflow 528, the low salt eluate having the protein-depleted DNA product (e.g., RCA product) is inputted (e.g., for flow through) into an anion exchange material 538 (e.g., anionic absorber membrane) having an interstitial space or pore size of 3 microns (micrometers) or greater to positively select for the DNA product without clogging. In certain embodiments, the ligand of the anion exchange material 538 may comprise n-benzyl-n-methyl ethanolamine, quaternary amine, diethylaminoethyl (DEAE), dimethylethanolamine (DMAE), or another anionic interaction ligand. Exemplary ion exchange media include DEAE-immobilized materials (for example CIMmultus®), DMAE-immobilized materials (for example Purexa® NAEX), quaternary amine-immobilized materials (for example Sartobind® Q), or immobilized N-benzyl-N-methylethanolamine media (for example Capto® Adhere). The protein-depleted high molecular weight DNA product (e.g., RCA product) binds to the anion exchange material 538 in low salt conditions (e.g., less than 1 M sodium chloride). Utilizing a pore size of less than 3 microns in the anion exchange material 538 results in clogging. The anion exchange material 538 with the bound DNA product is subject to washing with moderate salt (e.g., approximately 0.2 M sodium chloride) to remove undesirable RCA reaction components (e.g., dNTPs, excipients, etc.). Concentrated DNA product 540 (e.g., purified RCA product) is then eluted using 1 Molar sodium chloride. In preferred embodiments, the concentrated DNA product 540 is efficiently de-branched by trapping hyperbranched species within the pores of the anion exchange material 538.

In certain embodiments, the RCA product 530 from the amplification reactor may be applied directly to the anion exchange material 538 without subjecting to a prior affinity selection step to remove protein. In certain embodiments, depending on the use of the DNA product, protein components may not be removed (e.g., when the DNA product is not a vaccine) for certain downstream applications. In certain embodiments, the diluent buffer may be modified to remove protein components utilizing the anion exchange material 538 for a single-step chromatographic workflow.

The concentrated purified DNA product 540 in high concentration sodium chloride is then diluted 6 to 10× in water/buffer (as indicated by reference numeral 542) to obtain a formulated purified DNA product suitable for injection in physiological saline (e.g., at 0.154 molar solar chloride). The purified DNA product in the physiological saline is ready for fill-finish (e.g., aliquoting by the fill-finish module 18 in FIG. 1) as indicated by reference numeral 544. In certain embodiments, the purified DNA product is a vaccine. In preferred embodiments, the diluted purified DNA product 542 is substantially devoid of branched DNA by virtue of trapping hyperbranched species within the pores of the anion exchange material 538.

During the purification process, one or more sensors (e.g., arranged inline as part of a quality control system) may be utilized to measure one or more parameters to provide feedback to a controller that monitors the purification of the DNA product based on the feedback. The parameters may include conductivity, pH, viscosity, and/or absorbance at 260 nm and 280 nm. Conductivity enables confirming the appropriate salinity of wash and elution buffers. The parameter of pH enables confirming appropriate pH of wash and elution buffers and pH of the final product before fill-finish. OD260/280 enables fraction monitoring during elution and analyzing product purity/concentration. For example, the wash buffer used with the anion exchange material 538 must have lower salt and acidic/neural pH to maintain DNA in bound state. In contrast, the elution buffer used with the anion exchange material 538 must comprise higher salt and preferably a slightly alkaline pH to elute DNA in concentrated volumes. Also, OD260 monitoring may be utilized to coordinate DNA elution from the anion exchange material 538 with the corresponding buffer feed. After elution, OD260 quantitation may be utilized for exact dosing. Also, after elution, OD280 may be utilized to confirm maintenance of a low OD280 (protein) signal. Further, after elution, the ratio of OD260/280 may be utilized as a metric for purity of the DNA product (e.g., with an OD260/280 of 1.7-1.9 for pure DNA). Further pH may be utilized to confirm a pH of 7 or less of diluted purified DNA product for saline injection. Viscosity (e.g., via different pressure measurements) may be utilized for DNA molecular weight analysis since the purified DNA product is maintained in its original RCA concatemeric (polymer) state.

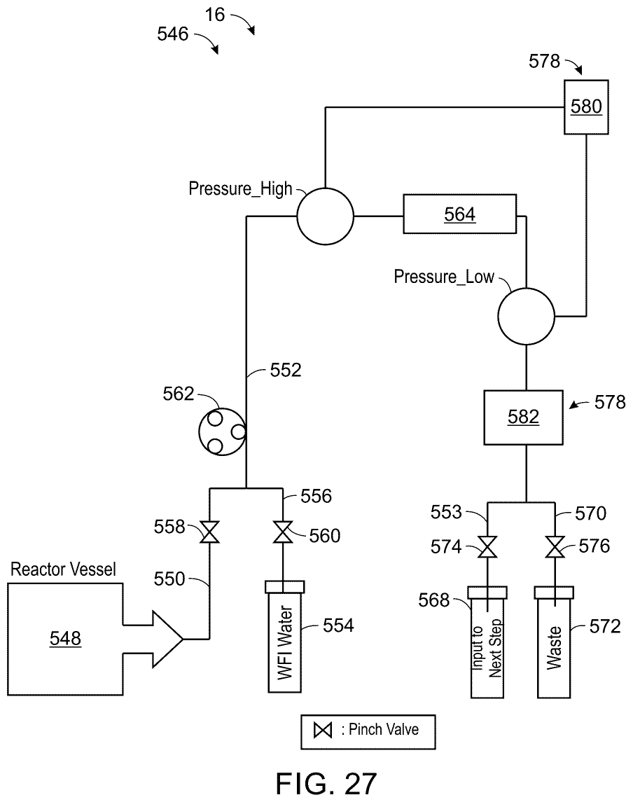

FIG. 27 is a schematic diagram of a fluid architecture 546 of a first portion (e.g., for protein removal) of an automated purification module 16 (e.g., for a large volume process for generating a single DNA product such as a vaccine). First, WFI water is introduced from a WFI supply 554 via a fluid pathway 556 to the fluid pathway 552 to condition a consumable chromatographic vessel 564 (e.g., remove storage buffer). Outflow from the consumable chromatographic vessel 564 is sent to waste along the fluid pathway 552 to fluid pathway 570 into a waste receptacle 572. Then a reaction volume (e.g., from an RCA reaction) is introduced (e.g., flowed along) to the purification module 16 from a reaction vessel 548 (e.g., of a DNA amplification module) via a fluid pathway 550 to a fluid pathway 552. In certain embodiments, and intermediate vessel may be utilized and contains the post-amplification reaction volume. In certain embodiments, the DNA product in reactor vessel 548 is diluted before being introduced into the purification module 16. In other embodiments, an intermediate mixing vessel may be utilized to dilute the DNA product. Valves 558, 560 (e.g., two-way pinch valves) are respectively disposed along the fluid pathways 550, 556 to respectively regulate flow of the reaction product and WFI water. A pump 562 (e.g., peristaltic pump) is disposed along the fluid pathway 552 to promote flow along the fluid pathway 552. In certain embodiments, separate pumps for the DNA product supply and the WFI supply may be used to control the dilution ratio as the fluids are delivered to the downstream components within the fluid architecture 546. After the removal of protein in consumable chromatographic vessel 564, the protein-depleted DNA product is flowed along the fluid pathway 552 to fluid pathway 553 to be inputted into the next purification step as indicated by reference numeral 568. Valves 574, 576 (e.g., two-way pinch valves) are respectively disposed along the fluid pathways 566, 570 to respectively regulate flow along these fluid pathways 566, 570. In preferred embodiments, the fluid architecture 546 comprises a single-use and functionally-closed consumable kit loaded into the purification module 16.

The consumable chromatographic vessel 564 disposed along the fluid pathway 552 contains affinity resin (e.g., immobilized heparin ligands) for depleting cations) protein (e.g., phi29 DNA polymerase) from the DNA reaction product. In certain embodiments, the affinity resin for depleting protein from the DNA reaction product is delivered within a storage buffer (e.g., 20% ethanol) that must be flushed away prior to use. For example, the consumable chromatographic vessel 564 may be a pre-packed heparin affinity column comprised of Heparin Sepharose® 6 Fast Flow and Capto® Heparin resin and stored in 20% ethanol.

Sensors 578 (e.g., as part of an inline quality control panel) are disposed along the fluid pathway 552. As depicted, the sensors 578 include a pressure sensor 580 (e.g., differential pressure gauge). The pressure sensor 580 measures the differential pressure across the consumable chromatographic vessel 564 (e.g., upstream and downstream) disposed along the fluid pathway 552. The pressure drop across the consumable chromatographic vessel 564 can be utilized to determine if a resin bed is clogged and fluid flow is being impeded. The sensors 578 also include an OD260 sensor 582 (e.g., quartz OD260 flow cell) to measure an optical absorbance of nucleic acids at 260 nanometers (nm) to determine a concentration of the nucleic acids. The OD260 sensor 582 has an optical pathway length of 0.2 mm. The various valves and pumps are responsive to control signals (e.g., to actuators) from a controller. The various sensors provide feedback to the controller.

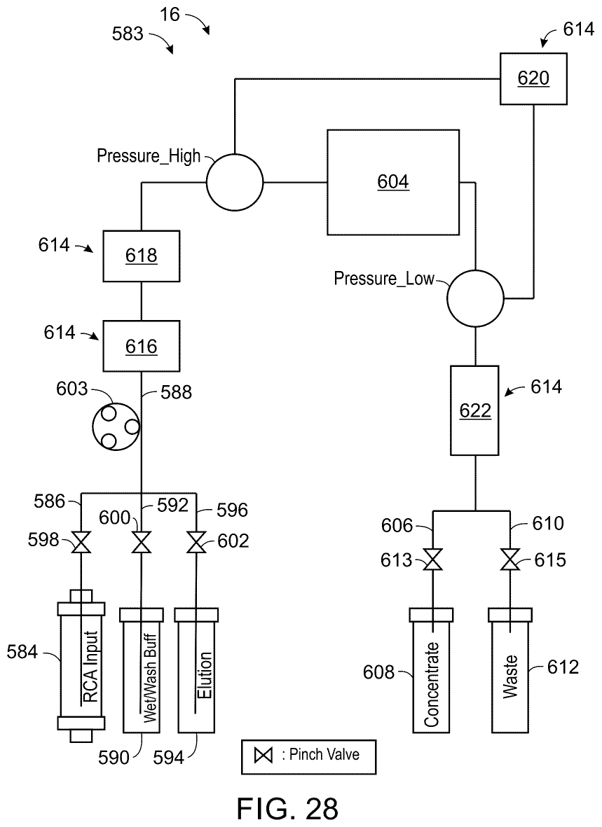

FIG. 28 is a schematic diagram of a fluid architecture 583 of a second portion (e.g., for ion exchange purification of protein-depleted DNA prior to product fill-finish) of an automated purification module 16 (e.g., for a large volume process for generating a single DNA product such as a vaccine). A reaction volume (e.g., containing material from an RCA reaction) 584 is introduced (e.g., flowed along) via a fluid pathway 586 to a fluid pathway 588 of the second portion of the purification module 16 as protein-depleted DNA (e.g., obtained from the first portion of the purification module 16 in FIG. 27). In certain embodiments, the reaction volume (e.g., in the form of diluted reaction volume) is introduced to the second portion of the purification module 16 without having gone through the first portion of the purification module 16 (i.e., protein removal). The fluid architecture 583 contains a consumable anion exchange column or membrane 604 for purifying and concentrating the DNA product. In preferred embodiments, the fluid architecture 583 comprises a single-use and functionally-closed consumable kit loaded into the purification module 16.

Wash buffer is introduced into fluid architecture 583 from a wash buffer supply 590 via a fluid pathway 592 to the fluid pathway 588 for washing steps (including removing storage buffer from the consumable anion exchange column or membrane 604). Elution buffer is also introduced from an elution buffer supply 594 via a fluid pathway 596 to the fluid pathway 588 for elution of the concentrated DNA product. Valves 598, 600, 602 are respectively disposed along the fluid pathways 586, 592, 596 (e.g., two-way pinch valves) to respectively regulate flow of the RCA-DNA-containing input, wash buffer, and elution buffer. A pump 603 (e.g., peristaltic pump) is disposed along the fluid pathway 588 to promote flow along the fluid pathway 588.

A consumable chromatographic column or membrane 604 (e.g., anion exchange column or membrane) having an interstitial space or pore size of 3 microns or greater is disposed along the fluid pathway 588 in a storage buffer. In certain embodiments, the cationic ligand of the consumable chromatographic column or membrane 604 may comprise n-benzyl-n-methyl ethanolamine, quaternary amine, diethylaminoethyl (DEAE), dimethylethanolamine (DMAE), or another anionic interaction ligand. Exemplary ion exchange consumable media include DEAE-immobilized materials (for example CIMmultus®), DMAE-immobilized materials (for example Purexa® NAEX), quaternary amine-immobilized materials (for example Sartobind® Q), or immobilized N-benzyl-N-methylethanolamine media (for example Capto® Adhere). The consumable chromatographic column or membrane 604 is subject to washing (e.g., a series of washes) with the wash buffer to remove storage buffer (e.g., 20% ethanol) and to additionally remove undesirable RCA reaction components (e.g., dNTPs, excipients, etc.). Concentrated DNA product (e.g., RCA product) is then eluted using the elution buffer.

The concentrated DNA product flows from the fluid pathway 588 to fluid pathway 606 to a receptacle 608. Waste flows from the fluid pathway 588 to fluid pathway 610 to a receptacle 612. Valves 613, 615 (e.g., two-way pinch valves) are respectively disposed along the fluid pathways 606, 610 to respectively regulate flow along these fluid pathways 606, 610.

Sensors 614 (e.g., as part of an inline quality control panel) are disposed along the fluid pathway 588. As depicted, the sensors 614 include a conductivity sensor 616 to measure a conductivity of a fluid flowing through fluid pathway 588. The sensors 614 also include a pH sensor (e.g., pH flow cell) to measure a pH of a fluid flowing through fluid pathway 588. The sensors 614 further include a pressure sensor 620 (e.g., differential pressure gauge). The pressure sensor 620 measures the differential pressure across the consumable chromatographic column or membrane 604 (e.g., measures upstream and downstream of the chromatographic column or membrane 604) disposed along the fluid pathway 588. The pressure drop across the consumable chromatographic column or membrane 604 can be utilized to determine if the column or membrane is clogged and the flow. is being impeded. The sensors 614 also include an OD260/OD280 sensor 622 (e.g., a OD260/280 flow cell) to measure an optical absorbance of nucleic acids at 260 nm and protein at 280 nm. The various valves and pumps are responsive to control signals (e.g., to actuators) from a controller. The various sensors provide feedback to the controller.

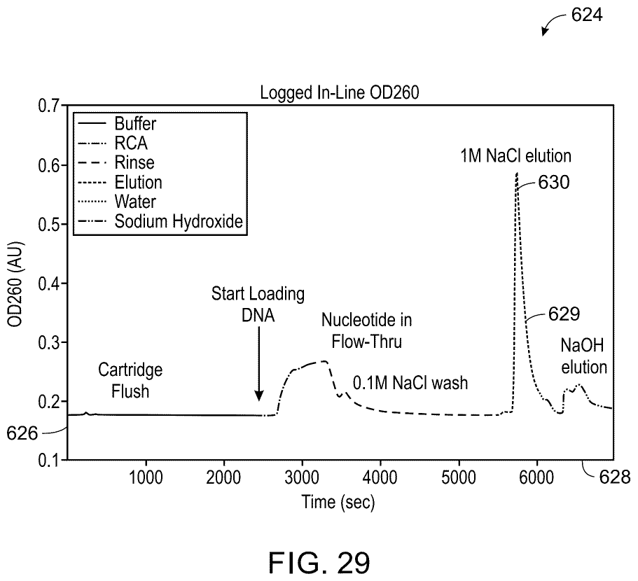

FIG. 29 further illustrates real-time in-line sensing during unit operation of a single-use kit for binding, washing, and eluting RCA DNA. In this example, a DEAE-immobilized ion exchange cartridge (CIMmultus 6 μm monolithic column) is flushed with water and equilibrated with wash buffer (20 mM Tris-HCl PH 7.0, 0.1M NaCl) prior to loading RCA DNA. The graph 624 includes a y-axis 626 representing measured OD260 (in absorbance units) downstream of the anion exchange membrane, while the x-axis 628 representing time (in seconds). Logged in-line OD260 nm data across a 0.2 μm pathlength depicts only baseline readings during this cartridge flush step. However, upon fluidic introduction of a completed RCA DNA reaction, in-line OD260 nm readings gradually rise as dNMPs and residual dNTPs flow thru the ion exchange cartridge and emerge on the filtrate side. Washing the cartridge with 10 column volumes of wash buffer returns in-line OD260 nm signal to baseline levels. However, upon fluidic introduction of elution buffer (1M NaCl), bound RCA DNA is eluted from the ion exchange cartridge and produces a large spike in OD260 nm signal at the filtrate side (peak 630). This purified DNA fraction is collected. The ion exchange cartridge can be optionally reconditioned (e.g. sodium hydroxide treatment) for subsequent re-use.

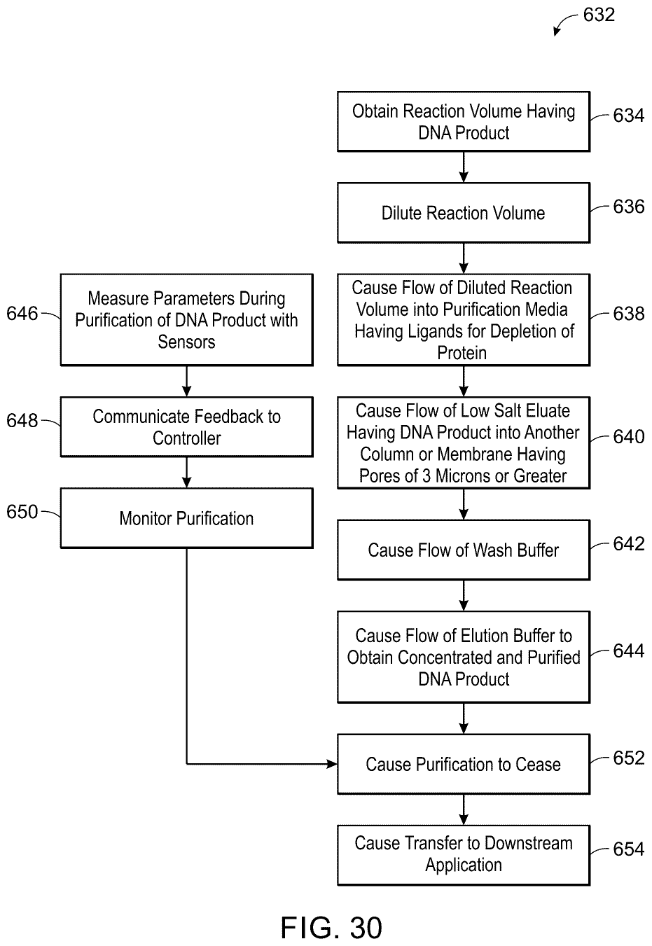

FIG. 30 is a flow chart of a method 632 for purification of a DNA product (e.g., from RCA reaction). One or more steps of the method 632 may be performed by processing circuitry and the purification module 16 of the system 10 in FIG. 1. One or more steps of the method 632 may be performed simultaneously and/or in a different order from that shown in FIG. 30.

The method 632 includes obtaining the reaction volume having the DNA product (e.g., RCA product) from an amplification reactor (e.g., of the DNA amplification module 14 in FIG. 1) (block 634). The method 632 also includes diluting the reaction volume with a buffer (e.g., at a ratio of 1:1) to form a diluted reaction volume (block 636). In certain embodiments, to enable the removal of protein (e.g., polymerase such as phi29) from the diluted reaction volume, the buffer includes EDTA (e.g., at a final concentration of 40 millimolar) and SDS (e.g., at a final concentration of 0.2 percent). In certain embodiments, the method 632 includes causing flow of the diluted reaction volume into a chromatographic vessel having affinity resin (e.g., immobilized heparin ligands) to deplete protein from the diluted reaction volume and generate a protein-depleted DNA product (e.g., RCA product) (block 638). This affinity purification results in greater than 99 percent removal of protein including phi29 DNA polymerase. The resin matrix of the chromatographic vessel doubles as a size exclusion matrix to remove the SDS, which avoids concentrating the SDS during the DNA purification step (as SDS is also negatively charged like DNA).

The method 632 also includes causing flow of the protein-depleted DNA product (e.g., RCA product) into another chromatographic column or membrane (e.g., anionic absorber membrane) having an interstitial space or pore size of 3 microns or greater to positively select for the DNA product using ion exchange (block 640). In certain embodiments, the protein removal may not be conducted first and the diluted reaction volume (e.g., without protein removal) may be caused to flow into the chromatographic column or membrane for ionic purification of DNA. In certain embodiments, the resin ligand of the chromatographic column or membrane for ionic purification of DNA may be n-benzyl-n-methyl ethanolamine, quaternary amine, diethylaminoethyl, dimethylethanolamine, or another anionic interaction ligand. In preferred embodiments, ionic purification of the protein-depleted DNA product efficiently de-branches the rolling circle amplification reaction product by trapping hyperbranched species within the pores.

The method 632 includes causing flow of wash buffer through the chromatographic column or membrane with the bound DNA product to remove the RCA reaction components (e.g., dNTPs, excipients, etc.) (block 642). The method 632 also includes causing flow of an elution buffer through the chromatographic column or membrane to obtain a concentrated and purified DNA product (block 644).

The method 632 further includes measuring one or more parameters during purification of the DNA product utilizing one or more sensors (block 646). The sensors and parameters are as described in FIGS. 27 and 28. The method 632 includes communicating feedback from the sensors to a controller having a memory and a processor (block 648). The method 632 also includes monitoring, via the controller, the purification of the DNA product based on the feedback (block 650). The method 632 further includes causing, via the controller, the purification to cease based on the feedback (block 652). The method 632 even further includes causing, via the controller, transfer of the purified DNA product to a downstream application (e.g., fill-finish module 18 in FIG. 1) (block 654). The DNA product may be diluted prior to transfer to the downstream application.

The present disclosure provides for systems and methods for purifying a DNA product from an RCA reaction. In ideal embodiments, the systems and methods for purifying the DNA product are configured to remove protein and generate a protein-depleted DNA product. Proteins to be removed from the RCA DNA reaction include DNA polymerase and any number of accessory enzymes, including primase, exonuclease, or ligase as non-limiting examples. It has been surprisingly discovered that phi29, a highly processive DNA polymerase, is very difficult to remove from DNA. It has been determined that only the conditions set forth in Example 1 are efficient at removing phi29, thereby generating a protein-depleted DNA product. It has been surprisingly discovered that many detergents fail to efficiently remove phi29 from DNA compared to SDS, as set forth in Example 2 using a heparin ligand. It is also surprisingly determined that buffer containing SDS and EDTA fail to efficiently remove phi29 from DNA when heparin ligand is replaced with sulfopropyl ligand of similar negative charge, as set forth in Example 2.

In one embodiment, the systems and methods configured to remove protein comprise a metal chelate affinity ligand. Non-limiting examples of metal chelate affinity ligands including those known in the art of immobilized metal chelate affinity chromatography (IMAC), including transition metals like nickel, cobalt, copper, or zinc. In another embodiment, the systems and methods configured to remove protein comprise a heparin ligand or heparin-mimicking ligand of similar net charge. It has been surprisingly discovered that the composition of the buffer used to form a diluted reaction volume also dictates the efficiency of ligand-based removal of phi29, as set forth in Example 1. Buffer containing sodium dodecyl sulfate is necessary for removal of phi29 polymerase from DNA onto IMAC ligand or heparin ligand. Buffers further comprising a chelating agent or high salt in combination with sodium dodecyl sulfate (SDS) are necessary and sufficient for efficient removal of phi29 polymerase, as set forth in Example 1. Non-limiting examples of chelating agent and salt include ethylenediaminetetraacetic acid (EDTA) and sodium chloride (NaCl), respectively.

In all embodiments, the ligands used to remove protein from DNA are immobilized onto one or more solid supports. Non-limiting examples of solid supports include chromatography resins, monolith matrices, gels, cellulosic membranes, or injected-molded materials. As such, the immobilized ligands may be implemented in both pre-packed and unpacked formats. One non-limiting embodiment of chromatography resin includes Heparin Sepharose 6 Fast Flow, as set forth in Example 3. It has been surprisingly discovered that accessory enzymes can be removed from DNA in the presence of SDS, EDTA, and immobilized heparin ligand, as set forth in Example 3. This non-limiting example utilized exonuclease I and exonuclease III to degrade non-circular template prior to initiating RCA, and all three enzymes are successfully removed from the DNA onto the resin, thereby generating a protein-depleted DNA product.

In further embodiments, the systems and methods for purifying DNA from RCA reactions are further configured to positively select protein-depleted DNA from reactants using anion exchange. Reactants to be removed from the RCA DNA reaction include deoxyribonucleoside monophosphates (dNMPs), inorganic pyrophosphate, reaction buffer, and any unincorporated deoxynucleotide triphosphates (dNTPs). During anion exchange, nucleic acids are initially bound under low-salt conditions and reactants are efficiently washed away from RCA DNA products by increasing the salt concentration. RCA DNA is then eluted in a purified form with high salt buffer. It has been surprisingly discovered that anion exchange methods can be configured to elute RCA DNA in a substantially de-branched state, as set forth in Example 4. The presence or absence of branched DNA produced from an RCA reaction is defined by the migration pattern produced under standard agarose gel electrophoresis. High-molecular weight branched DNA remains in the sample loading well, whereas de-branched DNA is able to migrate into the gel as a function of molecular weight and electrical field. Importantly, no enzymes are used to de-branch RCA DNA during anion exchange, so the removal of branched DNA is purely due to the physical nature of the anion exchange purification process.

In all embodiments, the ligands used to selectively purify DNA by anion exchange are immobilized onto one or more solid supports. Non-limiting examples of solid supports include chromatography resins, monolith matrices, gels, cellulosic membranes, or injected-molded materials. As such, the immobilized ligands may be implemented in both pre-packed and unpacked formats. One non-limiting embodiment of monolith matrix includes CIMmultus DEAE, as set forth in Example 4. In another non-limiting embodiment, the anion exchange matrix is Purexa DMAE or NAEX. In ideal embodiments, the pore size or interstitial space provided by the anion exchange material is at least 3 microns to avoid clogging.

### EXAMPLES

### Example 1: Protein Removal Efficiency from RCA DNA and Dependencies on Diluent Factors

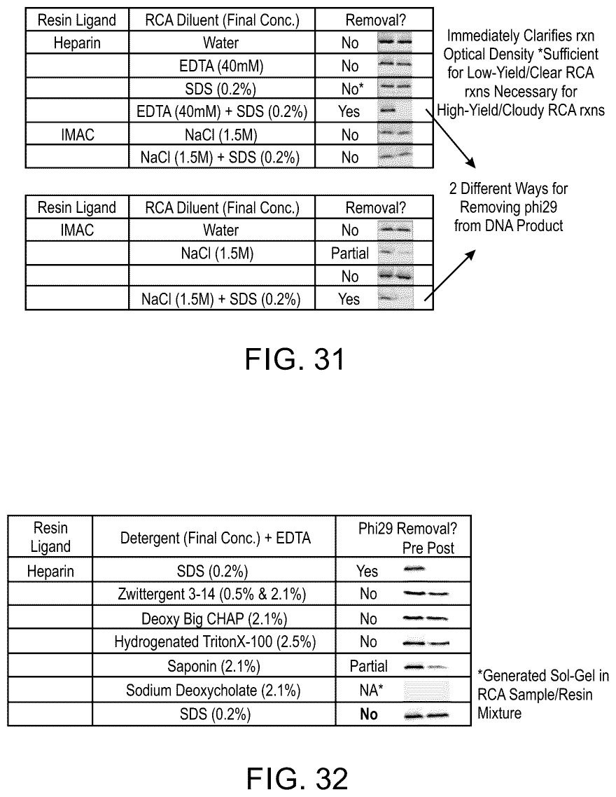

FIG. 31 summarizes how efficient phi29 may be removed from RCA DNA product as a function of resin type and diluent factors. For these experiments, RCA DNA products were amplified from input starting plasmid and completed RCA DNA reactions were diluted 1:1 with different diluents to achieve the listed final concentrations. These RCA DNA:diluent mixtures were then incubated with the listed resin types to assess affinity capture of phi29. Samples of RCA DNA were loaded before and after resin incubation onto SDS-PAGE gels and phi29 presence/absence was assessed using SYPRO Ruby gel stain. Exemplary gel images are presented in column 3 of FIG. 31. For wildtype phi29 enzyme, FIG. 31 demonstrates that heparin-immobilized resin (but not nickel-charged IMAC resin) efficiently depletes phi29 from RCA DNA in the presence of SDS-containing diluent. Inclusion of EDTA along with SDS is required for high-yielding RCA reactions, as the EDTA effectively chelates insoluble pyrophosphate salts and helps to improve phi29 removal from DNA in the presence of SDS and heparin-immobilized resin. In contrast, SDS alone is sufficient (without EDTA) for low-yielding RCA reactions that generate low concentrations of RCA DNA and produce no visible pyrophosphate byproduct. Those familiar in the art would recognize that EDTA is generally incompatible with metal-charged IMAC resin (due to chelation of the resin). Therefore, for recombinantly-tagged his6x-phi29 enzyme, FIG. 31 demonstrates that nickel-charged IMAC resin efficiently depletes his6-tagged phi29 RCA DNA in the presence of SDS and high-salt diluent. The high salt concentration does not chelate insoluble pyrophosphate but functionally replaces the role of EDTA in the presence of SDS detergent. In the absence of SDS detergent, high salt diluent only partially removes his6-tagged phi29 from RCA DNA product using nickel-charged IMAC resin.

FIG. 32 further demonstrates that SDS detergent is both necessary and sufficient for effective phi29 removal from RCA DNA in the presence of EDTA and heparin-immobilized resin. For these experiments, RCA DNA products were amplified from input starting plasmid and completed RCA DNA reactions were diluted 1:1 with EDTA diluent also containing different detergents to achieve the listed final concentrations. These RCA DNA diluent mixtures were then incubated with the listed resin types to assess affinity capture of wildtype phi29 enzyme. Samples of RCA DNA were loaded before and after resin incubation onto SDS-PAGE gels and phi29 presence/absence was assessed using SYPRO Ruby gel stain. Exemplary gel images are presented in column 3 of FIG. 32. FIG. 32 demonstrates that heparin-immobilized resin (but not sulfopropyl-immobilized resin) efficiently depletes phi29 from RCA DNA in the presence of SDS-containing EDTA diluent. Without ascribing to a particular mode of action, this data suggests that heparin-immobilized resin (but not sulfopropyl-immobilized resin) contains sufficient anionic character to out-compete phi29 enzyme from DNA. None of the other detergents tested in FIG. 32 (i.e. hydrogenated Triton X-100, Zwittergent 3-14, deoxy Big CHAP, sodium deoxycholate) could functionally replace SDS and assist in removing phi29 to undetectable levels. However, saponin (which is commonly used as a vaccine adjuvant) demonstrated some moderate ability to reduce phi29 from RCA DNA in the presence of EDTA and heparin-immobilized resin.

### Example 2: Removal of Proteins Beyond Phi29 from RCA DNA Reactions Using Heparin-Immobilized Resin in the Presence of EDTA and SDS

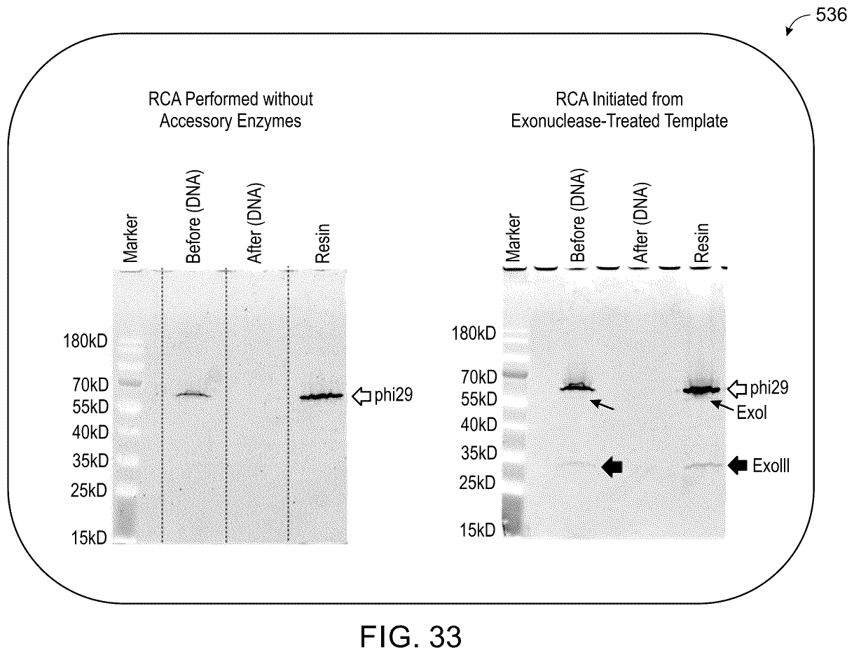

FIG. 33 demonstrates that heparin-immobilized resin is able to remove additional proteins (beyond phi29) from RCA DNA reactions in the presence of EDTA and SDS. For these experiments, RCA DNA products were amplified from an input starting plasmid that was optionally treated with exonucleases to digest non-circular DNA. One familiar in the art would recognize that Exo I and Exo III can be added to plasmids to remove damaged and/or non-circular species prior to RCA, but these exonucleases must be heat-denatured prior to RCA to avoid digesting the nascent RCA DNA product. Completed 20 ml RCA DNA reactions (with or without heat-denatured Exo I and Exo III) were diluted 1:1 with EDTA and SDS diluent and subsequently incubated with heparin-immobilized resin to assess affinity capture of wildtype phi29, Exo I, and Exo III enzymes. The heparin-immobilized resin was mixed with RCA DNA and allowed to gravity settle. Samples of RCA DNA were loaded before and after resin incubation onto SDS-PAGE gels and enzyme presence/absence was assessed using SYPRO Ruby gel stain. FIG. 33 demonstrates that all three enzymes were effectively removed from RCA DNA onto resin from input RCA DNA. No detectable protein content is visible in RCA DNA samples after resin incubation, thereby suggesting >99% removal efficiency (relative to RCA DNA samples before resin incubation).

### Example 3: Exemplary Purification Workflow for Protein Removal Using Heparin-Immobilized Resin, Followed by RCA DNA Concentration by Ion Exchange and Final Formulation in Physiological Saline

FIG. 26 outlines an exemplary purification workflow, wherein the first purification process uses SDS and EDTA diluents and heparin-immobilized resin to produce a protein-depleted RCA DNA feedstock for a second purification process that binds, concentrates, and elutes RCA DNA by ion exchange. Exemplary heparin-immobilized resin includes Heparin Sepharose® 6 Fast Flow and Capto® Heparin, which one familiar with the art would also recognize as size-exclusion media for removing small molecules (for example SDS and EDTA). During the subsequent process of binding and concentrating protein-depleted RCA DNA by ion exchange, impurities and byproducts from the RCA reaction (such as dNMPs, dNTPs, excipients, and buffer components) are further removed as filtrate or washed away upon introduction of low-salt buffers (for example 0.1M-0.2M NaCl). Exemplary ion exchange media include DEAE-immobilized materials (for example CIMmultus®), DMAE-immobilized materials (for example Purexa® NAEX), quaternary amine-immobilized materials (for example Sartobind® Q), or immobilized N-benzyl-N-methylethanolamine media (for example Capto® Adhere). Bound RCA DNA is eluted from these materials by increasing the salt concentration to high levels (for example, 1M NaCl), thereby yielding a purified product. Once eluted, the purified RCA DNA may be diluted to physiological salt levels (for example 0.15M NaCl) for downstream use.

### Example 4: Selective Purification of De-Branched RCA DNA Using Ion Exchange Materials

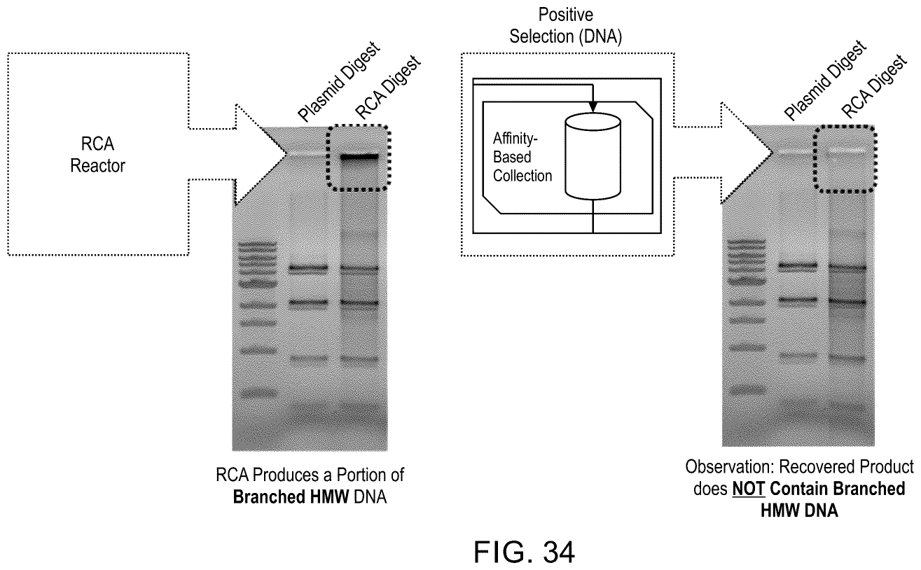

FIG. 34 demonstrates that ion exchange materials are capable of selectively purifying RCA DNA in a substantially de-branched state, as defined by the migration pattern produced under standard agarose gel electrophoresis. Initially, both high-molecular weight branched DNA species and lower-molecular weight DNA species are produced during the RCA reaction. FIG. 34 shows that high-molecular weight branched DNA remains in the gel loading well whereas low-molecular de-branched DNA migrates into the gel. When this feedstock is applied onto ion exchange materials (for example, DEAE CIMmultus or Purexa NAEX), followed by washing and elution protocols (e.g. 0.2M NaCl and 1M NaCl, respectively), only de-branched DNA is observed in the purified product which migrates into the ˜1% TBE agarose gel. Without ascribing to a particular hypothesis, this data suggests that branched high-molecular weight DNA is likely lost to the ion exchange material or perhaps requires higher concentrations of salt to elute efficiently. For the gel analysis provided in FIG. 34, all of the DNA samples (included starting plasmid template controls) have been digested with restriction enzymes to illustrate that branched high-molecular weight DNA persists, despite being exposed to restriction enzymes.

FIGS. 37A-C depict graphs 315, 317, and 319 of real time monitoring of RCA reactions under different conditions. 200 ul RCA reactions containing 800 uM dNTP were assembled containing SYBR green dye, and either 4 ng (square), 0.16 ng (circle) of a circular DNA template, or a no-template control (triangle). Each reaction was then divided into 180 ul and 18 ul, and inserted into a UV-transparent ½ well size, 96-well plate. This was then covered with a UV transparent seal and incubated at 23 degrees for 720 minutes, and monitored directly for fluorescence at 535 nm, OD at 260 nm, and OD at 304 nm at 10-minute increments. The raw values were then plotted to allow direct comparison of reaction kinetics. Both the 180 ul and the 18 ul reactions were monitored for fluorescence, but only the 18 ul results are shown as they had nearly identical kinetics. It can clearly be seen that the samples had a synchronous decrease in OD at 260 nm using a minimal path (˜1 mm) and increase in OD at 304 nm using a more typical path length (8 mm). This demonstrates that real time reaction kinetics of nucleic acid synthesis reactions can be monitored as a result of the different extinction coefficients of nucleotides, DNA and RNA at various wavelengths. Reactions that typically have too great an optical density to be monitored can be directly monitored using either a minimal path length at wavelengths between 260 nm and 285 nm, or with more standard path lengths at wavelengths between 295 nm and 310 nm.

Technical effects of the disclosed embodiments include providing systems and methods for improving nucleic acid synthesis. Technical effects of the disclosed embodiments include manufacturing nucleic acids with real time monitoring of key steps to ensure the output product meets desired quality metrics. Technical effects of the disclosed embodiments include providing multiple functional technology components (e.g., modules) that together function to generate the output product. Technical effects of the disclosed embodiments include providing an overall system that is configured to be field deployable to a location of need. Technical effects of the disclosed embodiments include providing a field deployable device that does not require highly skilled labor or a specialized operating environment. Technical effects of the disclosed embodiments include providing an overall system that utilizes an integrated workflow including enzymatic-based biosynthesis of DNA “seed” template, cell-free amplification of template DNA to rapidly produce bulk DNA quantities, and optional conversion to RNA product. Technical effects of the disclosed embodiments includes providing an entire workflow that includes quality control steps to produce GMP compliant fill/finish doses (e.g., for vaccine) and that is integrated within a user-friendly, portable structure that can be activated at a time of need (e.g., at the location of need) to produce hundreds of ready-to-use does for military or civilian first responder use (e.g., in less than 3 days).

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112 (f). However, for any claims containing elements designated in any other manner, it is intended that such elements are not to be interpreted under 35 U.S.C. 112 (f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system for purifying a deoxyribonucleic acid (DNA) product from a rolling circle amplification (RCA) reaction, comprising:
a DNA purification module configured to:
obtain a reaction volume having the DNA product from the RCA reaction;
dilute the reaction volume with a buffer to form a diluted reaction volume;
flow the diluted reaction volume through a first purification process configured to remove protein to generate a protein-depleted DNA product;
flow the protein-depleted DNA product through a second purification process configured to positively select for the DNA product using anion exchange; and
elute a purified DNA product from the second purification process.

2. The system of claim 1, wherein the second purification process is configured to cause elution of substantially de-branched DNA.

3. The system of claim 1, wherein the second purification process comprises an interstitial space or pore size of 3 microns or greater.

4. The system of claim 1, wherein the DNA purification module is configured to operate in a functionally-closed manner.

5. The system of claim 1, wherein the buffer comprises sodium dodecyl sulfate.

6. The system of claim 5, wherein the buffer further comprises a chelating agent, salt, or a mixture of both.

7. The system of claim 1, wherein the first purification process comprises a heparin ligand or heparin-mimicking ligand.

8. The system of claim 1, wherein the first purification process comprises a metal chelate affinity ligand.

9. The system of claim 1, wherein the DNA purification module comprises a controller having a memory and a processor, the DNA purification module comprises one or more sensors to measure one or more parameters during purification of the DNA product, the sensors communicate feedback to the controller, and the controller is configured to monitor the purification of the DNA product based on the feedback.

10. The system of claim 9, wherein the controller is configured to cause the purification to cease based on the feedback and to cause transfer of the purified DNA product to a fill-finish module.

11. The system of claim 9, wherein the one or more sensor parameters comprise optical absorbance at 260 nanometers, optical absorbance at 280 nanometers, pH, conductivity, and/or pressure.

12. The system of claim 9, wherein the one or more sensors are disposed inline.

13. The system of claim 1, wherein the DNA purification module is configured to purify the DNA product without utilizing an alcohol.

14. A method for purifying a deoxyribonucleic acid (DNA) product from a rolling circle amplification (RCA) reaction, comprising:
obtaining a reaction volume having the DNA product from the RCA reaction;
diluting the reaction volume with a buffer to form a diluted reaction volume; and
flowing the diluted reaction volume through a first purification process configured to remove protein and to generate a protein-depleted DNA product.

15. The method of claim 14, further comprising:
flowing the protein-depleted DNA product through a second purification process configured to positively select for the DNA product using anion exchange; and
eluting a purified DNA product from the second purification process.

16. The method of claim 15, wherein the second purification process is configured to cause elution of substantially de-branched DNA.

17. The method of claim 15, wherein the second purification process comprises an interstitial space or pore size of 3 microns or greater.

18. The method of claim 15, further comprising:
measuring one or more parameters during purification of the DNA product utilizing one or more sensors;
communicating feedback from sensors to a controller having a memory and a processor; and
monitoring, via the controller, the purification of the DNA product based on the feedback.

19. The method of claim 18, further comprising:
causing, via the controller, the purification to cease based on the feedback; and
causing, via the controller, transfer of the purified DNA product to a fill-finish module.

20. The method of claim 18, wherein the one or more parameters comprises optical absorbance at 260 nanometers, optical absorbance at 280 nanometers, pH, conductivity, and/or pressure.

21. The method of claim 18, wherein the sensors are disposed inline.

22. The method of claim 15, wherein purifying the DNA product occurs without utilizing an alcohol.

23. The method of claim 14, wherein the buffer comprises sodium dodecyl sulfate.

24. The method of claim 23, wherein the buffer further comprises a chelating agent, salt, or a mixture of both.

25. The method of claim 14, wherein the first purification process comprises a heparin ligand or heparin-mimicking ligand.

26. A non-transitory computer-readable medium, the computer-readable medium comprising processor-executable code that, when executed by a processing system, causes the processing system to:
obtain a reaction volume having a deoxyribonucleic acid (DNA) product from a rolling circle amplification reaction;
dilute the reaction volume with a buffer to form a diluted reaction volume;
flow the diluted reaction buffer through a first purification process configured to remove protein and to generate a protein-depleted DNA product;
flow the protein-depleted DNA product through a second purification process configured to positively select for the DNA product using anion exchange; and
elute of a purified DNA product from the second purification process.

27. The non-transitory computer-readable medium of claim 26, wherein the buffer comprises sodium dodecyl sulfate, and wherein the processor-executable code, when executed by the processing system, further causes the flow through the first and second purification processes prior to elution of the purified DNA product.
